(19) United States
(12) Patent Application Publication
(10) Pub. No.: US 2021/0052569 A1
(43) Pub. Date: Feb. 25, 2021

(54) TRANSDERMAL THERAPEUTIC SYSTEM CONTAINING NICOTINE AND SILICONE ACRYLIC HYBRID POLYMER

(71) Applicant: LTS LOHMANN THERAPIE-SYSTEME AG, Andemach (DE)

(72) Inventors: Michael LINN, Waldböckelheim (DE); Christoph SCHMITZ, Rheinbrohl (DE); Rolf BÖHM, Kleinmaischeid (DE); Marco EMGENBROICH, Rheinbach (DE)

(21) Appl. No.: 16/979,577

(22) PCT Filed: Mar. 11, 2019
(86) PCT No.: PCT/EP2019/056048

§ 371 Date: Sep. 10, 2020

(30) Foreign Application Priority Data
Mar. 13, 2018 (EP) 18161422.3

(51) Int. Cl. A61K 31/465; A61K 9/70

(52) U.S. Cl. CPC A61K 31/465; A61K 9/7069

(57) ABSTRACT

The present invention relates to transdermal therapeutic systems (TTS) for the transdermal administration of nicotine comprising a nicotine-containing layer structure, said nicotine-containing layer structure comprising A) a backing layer and B) a nicotine-containing layer, wherein the transdermal therapeutic system comprises a silicone acrylic hybrid polymer.

### TECHNICAL FIELD OF THE INVENTION

The present invention relates to a transdermal therapeutic system (TTS) for the transdermal administration of nicotine to the systemic circulation, and processes of manufacture, method of treatments and uses thereof.

### BACKGROUND OF THE INVENTION

Nicotine is an alkaloid found in the tobacco plant and other plants of the nightshade family and is present in dry tobacco at an amount of about 0.6-2.9% of dry weight. It is the main active substance of tobacco, acting both as stimulant and relaxant, and is highly addictive. Medicinally, nicotine is used in the treatment of nicotine dependency, as an aid in smoking cessation. It is currently available in a variety of dosage forms such as gums, lozenges, nasal sprays but also transdermal patches.

While dosage forms such as gums and sprays have a short onset and therefore are ideal to overcome cravings for a cigarette, they have a higher risk of promoting behavior typical for nicotine dependence. Transdermal patches on the other hand are easy to apply and maintain a nicotine blood level continuously and independent of any cravings, thus reducing the risk of promoting dependency behavior, and are able to ease withdrawal symptoms even in cases of heavy dependency.

However, nicotine is liquid at room temperature and due to the volatile nature of the drug substance, nicotine patches are difficult to formulate. Drying steps at higher temperature usually included in manufacturing processes of TTS lead to substantial drug loss. To avoid such loss, TTS with reservoir layers or matrix layers have been developed which can be manufactured e. g. by solvent-free systems using extrusion or by processes avoiding higher temperatures, e. g. by employing solvents with a low boiling point. These TTS however require a complex multilayer structure, either due to the reservoir layer necessitating a rate-controlling membrane and a skin contact layer providing adhesive properties, or due to supersaturation of the drug leading to drug segregation from the drug containing layer upon evaporating the solvent if not prevented by providing an additional layer (underlying the drug containing layer) into which the drug can diffuse.

The current TTS therefore are complicated and thus time-consuming and/or cost intensive to manufacture. In addition, it is difficult to achieve sufficient utilization of the active, since, as with all transdermal systems, the driving force of transdermal active release depends on the concentration gradient formed between the skin-contacting layer of the transdermal system and the skin, which is diminishing with time as the active amount in the transdermal system is reduced.

There is thus a need in the art for an improved transdermal therapeutic system for the transdermal administration of nicotine.

### OBJECTS AND SUMMARY OF THE INVENTION

It is an object of the present invention to provide a TTS overcoming the above-mentioned disadvantages of the currently available nicotine patches.

Thus, it is an object of the present invention to provide a TTS, and in particular a matrix-type TTS, for the transdermal administration of nicotine providing a permeation rate which is sufficient for achieving a therapeutically effective dose, while having a nicotine-containing layer structure of low complexity, which is consequently advantageous in terms of the ease and/or costs for the manufacture.

It is a further object of the present invention to provide a TTS for the transdermal administration of nicotine, wherein a constant release over an extended period of time is provided.

It is a further object of the present invention to provide a TTS for the transdermal administration of nicotine with high active utilization.

It is an object of certain embodiments of the present invention to provide a TTS for the transdermal administration of nicotine, wherein therapeutically effective amounts of nicotine are provided for 1 day by said transdermal therapeutic system during an administration period to the skin of the patient of 1 day, allowing a once a day exchange of the TTS in an around the clock treatment.

These objects and others are accomplished by the present invention, which according to one first aspect relates to a transdermal therapeutic system for the transdermal administration of nicotine comprising a nicotine-containing layer structure, said nicotine-containing layer structure comprising:
A) a backing layer; and
B) a nicotine-containing layer;
   wherein the transdermal therapeutic system comprises a silicone acrylic hybrid polymer, and
   wherein the nicotine-containing layer structure comprises at least 0.8 mg/cm² nicotine.

According to a second aspect, the present invention relates to a transdermal therapeutic system for the transdermal administration of nicotine comprising a nicotine-containing layer structure, said nicotine-containing layer structure comprising:
A) a backing layer; and
B) a nicotine-containing layer comprising
   1. nicotine; and
   2. the silicone acrylic hybrid polymer.

According to a third aspect, the present invention relates to a transdermal therapeutic system for the transdermal administration of nicotine comprising a nicotine-containing self-adhesive layer structure comprising:
A) a backing layer; and
B) a nicotine-containing pressure-sensitive adhesive layer comprising:
   1. nicotine included in the form of the free base in an amount of 4% to 10% of the nicotine-containing pressure-sensitive adhesive layer; and
   2. a silicone acrylic hybrid pressure-sensitive adhesive in an amount of from 90 to 96% of the nicotine-containing pressure-sensitive adhesive layer;
      wherein the area weight of the nicotine-containing pressure-sensitive adhesive layer ranges from 100 to 250 g/m²,
      wherein the silicone acrylic hybrid pressure-sensitive adhesive in the nicotine-containing pressure-sensitive adhesive layer contains a continuous, silicone external phase and a discontinuous, acrylic internal phase,
      wherein the self-adhesive layer structure does not comprise an additional skin contact layer.

According to a fourth aspect, the present invention relates to a transdermal therapeutic system for the transdermal administration of nicotine comprising a nicotine-containing self-adhesive layer structure comprising:
A) a backing layer; and
B) a nicotine-containing pressure-sensitive adhesive layer comprising:
   1. nicotine included in the form of the free base in an amount of 6% to 10% of the nicotine-containing pressure-sensitive adhesive layer; and
   2. a silicone acrylic hybrid pressure-sensitive adhesive in an amount of from 90 to 94% of the nicotine-containing pressure-sensitive adhesive layer;
      wherein the area weight of the nicotine-containing pressure-sensitive adhesive layer ranges from 90 to 150 g/m², and
      wherein the silicone acrylic hybrid pressure-sensitive adhesive in the nicotine-containing pressure-sensitive adhesive layer contains a continuous, silicone external phase and a discontinuous, acrylic internal phase,
      wherein the self-adhesive layer structure comprises an additional skin contact layer with an area weight of from 80 to 140 g/m².

According to yet another aspect, the invention relates to a process for manufacturing a nicotine-containing pressure-sensitive adhesive layer comprising the steps of:
1) combining at least the components nicotine and a silicone acrylic hybrid polymer in a solvent to obtain a coating composition;
2) coating the coating composition onto a backing layer or a release liner; and
3) drying the coated coating composition to form the nicotine-containing pressure-sensitive adhesive layer.

### Definitions

Within the meaning of this invention, the term “transdermal therapeutic system” (TTS) refers to a system by which the active agent (e. g. nicotine) is administered to the systemic circulation via transdermal delivery and refers to the entire individual dosing unit that is applied, after removing an optionally present release liner, to the skin of a patient, and which comprises a therapeutically effective amount of active agent in an active agent-containing layer structure and optionally an additional adhesive overlay on top of the active agent-containing layer structure. The active agent-containing layer structure may be located on a release liner (a detachable protective layer), thus, the TTS may further comprise a release liner. Within the meaning of this invention, the term “TTS” in particular refers to systems providing transdermal delivery, excluding active delivery for example via iontophoresis or microporation. Transdermal therapeutic systems may also be referred to as transdermal drug delivery systems (TDDS) or transdermal delivery systems (TDS).

Within the meaning of this invention, the term “nicotine-containing layer structure” refers to the layer structure containing a therapeutically effective amount of nicotine and comprises a backing layer and at least one active agent-containing layer. Preferably, the nicotine-containing layer structure is a nicotine-containing self-adhesive layer structure.

Within the meaning of this invention, the term “therapeutically effective amount” refers to a quantity of active agent in the TTS sufficient to ease nicotine withdrawal symptoms, if administered by the TTS to a patient. A TTS usually contains more active in the system than is in fact provided to the skin and the systemic circulation. This excess amount of active agent is usually necessary to provide enough driving force for the delivery from the TTS to the systemic circulation.

Within the meaning of this invention, the terms “active”, “active agent”, and the like, as well as the term “nicotine” refer to nicotine in any pharmaceutically acceptable chemical and morphological form and physical state. These forms include without limitation nicotine in its free base form, protonated or partially protonated nicotine, nicotine salts and in particular acid addition salts formed by addition of an inorganic or organic acid such as nicotine bitartrate or nicotine hydrochloride, solvates, hydrates, clathrates, complexes and so on. As nicotine in its pure free base form is liquid at room temperature, the forms include nicotine in liquid form or nicotine (e.g. nicotine salts) in the form of particles which may be micronized, crystalline and/or amorphous, and any mixtures of the aforementioned forms. The nicotine, where contained in a medium such as a solvent, may be dissolved or dispersed or in part dissolved and in part dispersed.

When nicotine is mentioned to be used in a particular form in the manufacture of the TTS, this does not exclude interactions between this form of nicotine and other ingredients of the nicotine-containing layer structure, e.g. salt formation or complexation, in the final TTS. This means that, even if nicotine is included in its free base form, it may be present in the final TTS in protonated or partially protonated form or in the form of an acid addition salt, or, if it is included in the form of a salt, parts of it may be present as free base in the final TTS. Unless otherwise indicated, in particular the amount of nicotine in the layer structure relates to the amount of nicotine included in the TTS during manufacture of the TTS and is calculated based on nicotine in the form of the free base. E. g., when a) 0.1 mmol (equal to 16.2 mg) nicotine base or b) 0.1 mmol (equal to 46.2 mg) nicotine bitartrate is included in the TTS during manufacture, the amount of nicotine in the layer structure is, within the meaning of the invention, in both cases 0.1 mmol or 16.2 mg.

The nicotine starting material included in the TTS during manufacture of the TTS may be in the form of particles. nicotine may e. g. be present in the active agent-containing layer structure in the form of particles and/or dissolved.

Within the meaning of this invention, the term “particles” refers to a solid, particulate material comprising individual particles, the dimensions of which are negligible compared to the material. In particular, the particles are solid, including plastic/deformable solids, including amorphous and crystalline materials.

Within the meaning of this invention, the term “dispersing” refers to a step or a combination of steps wherein a starting material (e. g. nicotine) is not totally dissolved. Dispersing in the sense of the invention comprises the dissolution of a part of the starting material, depending on the solubility of the starting material (e. g. the solubility of nicotine in the coating composition).

There are two main types of TTS for active agent delivery, i.e. matrix-type TTS and reservoir-type TTS. The release of the active agent in a matrix-type TTS is mainly controlled by the matrix including the active agent itself. In contrast thereto, a reservoir-type TTS typically needs a rate-controlling membrane controlling the release of the active agent. In principle, also a matrix-type TTS may contain a rate-controlling membrane. However, matrix-type TTS are advantageous in that, compared to reservoir-type TTS, usually no rate determining membranes are necessary and no dose dumping can occur due to membrane rupture. In summary, matrix-type transdermal therapeutic systems (TTS) are less complex in manufacture and easy and convenient to use by patients.

Within the meaning of this invention, “matrix-type TTS” refers to a system or structure wherein the active is homogeneously dissolved and/or dispersed within a polymeric carrier, i. e. the matrix, which forms with the active agent and optionally remaining ingredients a matrix layer. In such a system, the matrix layer controls the release of the active agent from the TTS. Preferably, the matrix layer has sufficient cohesion to be self-supporting so that no sealing between other layers is required. Accordingly, the nicotine-containing layer may in one embodiment of the invention be a nicotine-containing matrix layer, wherein the nicotine is homogeneously distributed within a polymer matrix. In certain embodiments, the nicotine-containing matrix layer may comprise two nicotine-containing matrix layers, which may be laminated together. Matrix-type TTS may in particular be in the form of a “drug-in-adhesive”-type TTS referring to a system wherein the active is homogeneously dissolved and/or dispersed within a pressure-sensitive adhesive matrix. In this connection, the nicotine-containing matrix layer may also be referred to as nicotine-containing pressure sensitive adhesive layer or nicotine-containing pressure sensitive adhesive matrix layer. A TTS comprising the active agent dissolved and/or dispersed within a polymeric gel, e. g. a hydrogel, is also considered to be of matrix-type in accordance with present invention.

TTS with a liquid active agent-containing reservoir are referred to by the term “reservoir-type TTS”. In such a system, the release of the active agent is preferably controlled by a rate-controlling membrane. In particular, the reservoir is sealed between the backing layer and the rate-controlling membrane. Accordingly, the nicotine-containing layer may in one embodiment be a nicotine-containing reservoir layer, which preferably comprises a liquid reservoir comprising the nicotine. Furthermore, the reservoir-type TTS typically additionally comprises a skin contact layer, wherein the reservoir layer and the skin contact layer may be separated by the rate-controlling membrane. In the reservoir layer, the active agent is preferably dissolved in a solvent such as ethanol or water or in silicone oil. The skin contact layer typically has adhesive properties.

Reservoir-type TTS are not to be understood as being of matrix-type within the meaning of the invention. However, microreservoir TTS (biphasic systems having deposits (e. g. spheres, droplets) of an inner active-containing phase dispersed in an outer polymer phase), considered in the art to be a mixed from of a matrix-type TTS and a reservoir-type TTS that differ from a homogeneous single phase matrix-type TTS and a reservoir-type TTS in the concept of drug transport and drug delivery, are considered to be of matrix-type within the meaning of the invention. The sizes of microreservoir droplets can be determined by an optical microscopic measurement (for example by Leica MZ16 including a camera, for example Leica DSC320) by taking pictures of the microreservoirs at different positions at an enhancement factor between 10 and 400 times, depending on the required limit of detection. By using imaging analysis software, the sizes of the microreservoirs can be determined.

Within the meaning of this invention, the term “nicotine-containing layer” refers to a layer containing the nicotine and providing the area of release. The term covers nicotine-containing matrix layers and nicotine-containing reservoir layers. If the nicotine-containing layer is a nicotine-containing matrix layer, said layer is present in a matrix-type TTS. If the polymer is a pressure-sensitive adhesive, the matrix layer may also represent the adhesive layer of the TTS, so that no additional skin contact layer is present. Alternatively, an additional skin contact layer may be present as adhesive layer, and/or an adhesive overlay is provided. The additional skin contact layer is typically manufactured such that it is active agent-free. However, due to the concentration gradient, the active agent will migrate from the matrix layer to the additional skin contact layer over time, until equilibrium is reached. The additional skin contact layer may be present on the nicotine-containing matrix layer or separated from the nicotine-containing matrix layer by a membrane, preferably a rate controlling membrane. Preferably, the nicotine-containing matrix layer has sufficient adhesive properties, so that no additional skin contact layer is present. If the nicotine-containing layer is a nicotine-containing reservoir layer, said layer is present in a reservoir-type TTS, and the layer comprises the nicotine in a liquid reservoir. In addition, an additional skin contact layer is preferably present, in order to provide adhesive properties. Preferably, a rate-controlling membrane separates the reservoir layer from the additional skin contact layer. The additional skin contact layer can be manufactured such that it is active agent-free or active agent-containing. If the additional skin contact layer is free of active agent the active agent will migrate, due to the concentration gradient, from the reservoir layer to the skin contact layer over time, until equilibrium is reached. Additionally an adhesive overlay may be provided.

As used herein, the nicotine-containing layer is preferably a nicotine-containing matrix layer, and it is referred to the final solidified layer. Preferably, a nicotine-containing matrix layer is obtained after coating and drying the solvent-containing coating composition as described herein. Alternatively a nicotine-containing matrix layer is obtained after melt-coating and cooling. The nicotine-containing matrix layer may also be manufactured by laminating two or more such solidified layers (e. g. dried or cooled layers) of the same composition to provide the desired area weight. The matrix layer may be self-adhesive (in the form of a pressure sensitive adhesive matrix layer), or the TTS may comprise an additional skin contact layer of a pressure sensitive adhesive for providing sufficient tack. Preferably, the matrix layer is a pressure sensitive adhesive matrix layer. Optionally, an adhesive overlay may be present.

Within the meaning of this invention, the term “pressure-sensitive adhesive” (also abbreviated as “PSA”) refers to a material that in particular adheres with finger pressure, is permanently tacky, exerts a strong holding force and should be removable from smooth surfaces without leaving a residue. A pressure sensitive adhesive layer, when in contact with the skin, is “self-adhesive”, i. e. provides adhesion to the skin so that typically no further aid for fixation on the skin is needed. A “self-adhesive” layer structure includes a pressure sensitive adhesive layer for skin contact which may be provided in the form of a pressure sensitive adhesive matrix layer or in the form of an additional layer, i.e. a pressure sensitive adhesive skin contact layer. An adhesive overlay may still be employed to advance adhesion. The pressure-sensitive adhesive properties of a pressure-sensitive adhesive depend on the polymer or polymer composition used.

Within the meaning of this invention, the term “silicone acrylic hybrid polymer” refers to a polymerization product including repeating units of a silicone sub-species and an acrylate-sub species. The silicone acrylic hybrid polymer thus comprises a silicone phase and an acrylic phase. The term “silicone acrylic hybrid” is intended to denote more than a simple blend of a silicone-based sub-species and an acrylate-based sub-species. Instead, the term denotes a polymerized hybrid species that includes silicone-based sub-species and acrylate-based sub-species that have been polymerized together. The silicone acrylic hybrid polymer may also be referred to as a “silicone acrylate hybrid polymer” as the terms acrylate and acrylic are generally used interchangeably in the context of the hybrid polymers used in the present invention.

Within the meaning of this invention, the term “silicone acrylic hybrid pressure-sensitive adhesive” refers to a silicone acrylic hybrid polymer in the form of a pressure-sensitive adhesive. Silicone acrylic hybrid pressure-sensitive adhesives are described, for example, in EP 2 599 847 and WO 2016/130408. Examples of silicone acrylic hybrid pressure-sensitive adhesives include the PSA series 7-6100 and 7-6300 manufactured and supplied in n-heptane or ethyl acetate by Dow Corning (7-610X and 7-630X; X=1 n-heptane-based/X=2 ethyl acetate-based). It was found that, depending on the solvent in which the silicone acrylic hybrid PSA is supplied, the arrangement of the silicone phase and the acrylic phase providing a silicone or acrylic continuous external phase and a corresponding discontinuous internal phase is different. If the silicone acrylic hybrid PSA is supplied in n-heptane, the composition contains a continuous, silicone external phase and a discontinuous, acrylic internal phase. If the silicone acrylic hybrid PSA composition is supplied in ethyl acetate, the composition contains a continuous, acrylic external phase and a discontinuous, silicone internal phase.

Within the meaning of this invention, the term “non-hybrid polymer” is used synonymously for a polymer which does not include a hybrid species. Preferably, the non-hybrid polymer is a pressure-sensitive adhesive (e. g. a silicone- or acrylate-based pressure-sensitive adhesives).

Within the meaning of this invention, the term “silicon-containing pressure-sensitive adhesive composition comprising acrylate or methacrylate functionality” comprises the condensation reaction product of a silicone resin, a silicone polymer, and a silicon-containing capping agent which provides said acrylate or methacrylate functionality. It is to be understood that the silicon-containing pressure-sensitive adhesive composition comprising acrylate or methacrylate functionality can include only acrylate functionality, only methacrylate functionality, or both acrylate functionality and methacrylate functionality.

As used herein, an active agent-containing matrix layer is a layer containing the active agent dissolved or dispersed in at least one polymer, or containing the active agent dissolved in a solvent to form an active agent-solvent mixture that is dispersed in the form of deposits (in particular droplets) in at least one polymer. Preferably, the at least one polymer is a polymer-based pressure-sensitive adhesive (e. g. a silicone acrylic hybrid pressure-sensitive adhesive). Within the meaning of this invention, the term “pressure-sensitive adhesive layer” refers to a pressure-sensitive adhesive layer obtained from a solvent-containing adhesive coating composition after coating on a film and evaporating the solvents.

Within the meaning of this invention, the term “skin contact layer” refers to the layer included in the nicotine-containing layer structure to be in direct contact with the skin of the patient during administration. This may be the nicotine-containing layer. When the TTS comprises an additional skin contact layer, the other layers of the nicotine-containing layer structure do not contact the skin and do not necessarily have self-adhesive properties. As outlined above, an additional skin contact layer attached to the nicotine-containing layer may over time absorb parts of the active agent. An additional skin contact layer may be used to enhance adherence. The sizes of an additional skin contact layer and the nicotine-containing layer are usually coextensive and correspond to the area of release. However, the area of the additional skin contact layer may also be greater than the area of the nicotine-containing layer. In such a case, the area of release still refers to the area of the nicotine-containing layer.

Within the meaning of this invention, the term “area weight” refers to the dry weight of a specific layer, e.g. of the matrix layer, provided in g/m². The area weight values are subject to a tolerance of ±10%, preferably ±7.5%, due to manufacturing variability.

If not indicated otherwise “%” refers to weight-%.

Within the meaning of this invention, the term “polymer” refers to any substance consisting of so-called repeating units obtained by polymerizing one or more monomers, and includes homopolymers which consist of one type of monomer and copolymers which consist of two or more types of monomers. Polymers may be of any architecture such as linear polymers, star polymer, comb polymers, brush polymers, of any monomer arrangements in case of copolymers, e.g. alternating, statistical, block copolymers, or graft polymers. The minimum molecular weight varies depending on the polymer type and is known to the skilled person. Polymers may e.g. have a molecular weight above 2000, preferably above 5000 and more preferably above 10,000 Dalton. Correspondingly, compounds with a molecular weight below 2000, preferably below 5000 or more preferably below 10,000 Dalton are usually referred to as oligomers.

Within the meaning of this invention, the term “cross-linking agent” refers to a substance which is able to cross-link functional groups contained within the polymer.

Within the meaning of this invention, the term “adhesive overlay” refers to a self-adhesive layer structure that is free of active agent and larger in area than the active agent-containing structure and provides additional area adhering to the skin, but no area of release of the active agent. It enhances thereby the overall adhesive properties of the TTS. The adhesive overlay comprises a backing layer that may provide occlusive or non-occlusive properties and an adhesive layer. Preferably, the backing layer of the adhesive overlay provides non-occlusive properties.

Within the meaning of this invention, the term “backing layer” refers to a layer which supports the active agent-containing layer or forms the backing of the adhesive overlay. At least one backing layer in the TTS and usually the backing layer of the active agent-containing layer is substantially impermeable to the active agent contained in the layer during the period of storage and administration and thus prevents active loss or cross-contamination in accordance with regulatory requirements. Preferably, the backing layer is also occlusive, meaning substantially impermeable to water and water-vapor. Suitable materials for a backing layer include polyethylene terephthalate (PET), polyethylene (PE), ethylene vinyl acetate-copolymer (EVA), polyurethanes, and mixtures thereof. Suitable backing layers are thus for example PET laminates, EVA-PET laminates and PE-PET laminates.

The TTS according to the present invention can be characterized by certain parameters as measured in an in vitro skin permeation test.

The in vitro permeation test is performed in a Franz diffusion cell, with human or animal skin and preferably with dermatomed split-thickness human skin with a thickness of 800 μm and an intact epidermis, and with phosphate buffer pH 5.5 or 7.4 as receptor medium (32° C. with 0.1% saline azide) with or without addition of a maximum of 40 vol-% organic solvent e.g. ethanol, acetonitrile, isopropanol, dipropylenglycol, PEG 400 so that a receptor medium may e.g. contain 60 vol-% phosphate buffer pH 5.5, 30 vol-% dipropylenglycol and 10 vol-% acetonitrile.

Where not otherwise indicated, the in vitro permeation test is performed with dermatomed split-thickness human skin with a thickness of 800 μm and an intact epidermis, and with phosphate buffer pH 5.5 as receptor medium (32° C. with 0.1% saline azide). The amount of active permeated into the receptor medium is determined in regular intervals using a validated HPLC method with a UV photometric detector by taking a sample volume. The receptor medium is completely or in part replaced by fresh medium when taking the sample volume, and the measured amount of active permeated relates to the amount permeated between the two last sampling points and not the total amount permeated so far.

Thus, within the meaning of this invention, the parameter “permeated amount” is provided in μg/cm² and relates to the amount of active permeated in a sample interval at certain elapsed time. E.g., in an in vitro permeation test as described above, wherein the amount of active permeated into the receptor medium has been e.g. measured at hours 0, 2, 4, 8, 12 and 24, the “permeated amount” of active can be given e.g. for the sample interval from hour 8 to hour 12 and corresponds to the measurement at hour 12, wherein the receptor medium has been exchanged completely at hour 8.

The permeated amount can also be given as a “cumulative permeated amount”, corresponding to the cumulated amount of active permeated at a certain point in time. E. g., in an in vitro permeation test as described above, wherein the amount of active permeated into the receptor medium has been e.g. measured at hours 0, 2, 4, 8, 12 and 24, the “cumulative permeated amount” of active at hour 12 corresponds to the sum of the permeated amounts from hour 0 to hour 2, hour 2 to hour 4, hour 4 to hour 8 and hour 8 to hour 12.

Within the meaning of this invention, the parameter “skin permeation rate” for a certain sample interval at certain elapsed time is provided in μg/cm²-hr and is calculated from the permeated amount in said sample interval as measured by in vitro permeation test as described above in μg/cm², divided by the hours of said sample interval. E. g. the skin permeation rate in an in vitro permeation test as described above, wherein the amount of active permeated into the receptor medium has been e.g. measured at hours 0, 2, 4, 8, 12 and 24, the “skin permeation rate” at hour 12 is calculated as the permeated amount in the sample interval from hour 8 to hour 12 divided by 4 hours.

A “cumulative skin permeation rate” can be calculated from the respective cumulative permeated amount by dividing the cumulative permeated amount by the elapsed time. E.g. in an in vitro permeation test as described above, wherein the amount of active permeated into the receptor medium has been e.g. measured at hours 0, 2, 4, 8, 12 and 24, the “cumulative skin permeation rate” at hour 12 is calculated as the cumulative permeated amount for hour 12 (see above) divided by 12 hours.

Within the meaning of this invention, the above parameters “permeated amount” and “skin permeation rate” (as well as “cumulative permeated amount” and “cumulative skin permeation rate”) refer to mean values calculated from at least 3 in vitro permeation test experiments. Where not otherwise indicated, the standard deviation (SD) of these mean values refer to a corrected sample standard deviation, calculated using the formula:

$$SD = \sqrt{\frac{1}{n - 1} {\sum }_{i = 1}^{n} {( {x}_{i} - \overline{x} )}^{2}}$$

wherein n is the sample size, {x₁, x₂, . . . xₙ} are the observed values and x̅ is the mean value of the observed values.

The TTS according to the present invention can also be characterized by certain parameters as measured in an in vivo clinical study.

Within the meaning of this invention, the parameter “mean release rate” refers to the mean release rate in μg/hr or in mg/day over the period of administration (e. g., 1 to 7 days) by which the active agent is released through the human skin into the systemic circulation and is based on the AUC obtained over said period of administration in a clinical study.

Within the meaning of this invention, the term “extended period of time” relates to a period of at least or about 6 hours, at least or about 8 hours, at least 12 hours, at least or about 16 hours or at least or about 24 hours.

Within the meaning of this invention, the term “room temperature” refers to the unmodified temperature found indoors in the laboratory where the experiments are conducted and usually lies within 15 to 35° C., preferably about 18 to 25° C.

Within the meaning of this invention, the term “patient” refers to a subject who has presented a clinical manifestation of a particular symptom or symptoms suggesting the need for treatment, who is treated preventatively or prophylactically for a condition, or who has been diagnosed with a condition to be treated.

Within the meaning of this invention the term “pharmacokinetic parameters” refers to parameters describing the blood plasma curve, e.g. Cₘₐₓ, Cₜ and AUCₜ₁₋ₜ₂ obtained in a clinical study, e.g. by single-dose, multi-dose or steady state administration of the active agent-containing TTS, e.g. the nicotine TTS to healthy human subjects. The pharmacokinetic parameters of the individual subjects are summarized using arithmetic and geometric means, e. g. a mean Cₘₐₓ, a mean AUCt and a mean AUCINF, and additional statistics such as the respective standard deviations and standard errors, the minimum value, the maximum value, and the middle value when the list of values is ranked (Median). In the context of the present invention, pharmacokinetic parameters, e.g. the Cₘₐₓ, Cₜ and AUCₜ₁₋ₜ₂ refer to geometric mean values if not indicated otherwise. It cannot be precluded that the absolute mean values obtained for a certain TTS in a clinical study vary to a certain extent from study to study. To allow a comparison of absolute mean values between studies, a reference formulation, e.g. in the future any product based on the invention, may be used as internal standard. A comparison of the AUC per area of release of the respective reference product in the earlier and later study can be used to obtain a correction factor to take into account differences from study to study.

Clinical studies according to the present invention refer to studies performed in full compliance with the International Conference for Harmonization of Clinical Trials (ICH) and all applicable local Good Clinical Practices (GCP) and regulations.

Within the meaning of this invention, the term “healthy human subject” refers to a male or female subject with a body weight ranging from 55 kg to 100 kg and a body mass index (BMI) ranging from 18 to 29.4 and normal physiological parameters, such as blood pressure, etc. Healthy human subjects for the purposes of the present invention are selected according to inclusion and exclusion criteria which are based on and in accordance with recommendations of the ICH.

Within the meaning of this invention, the term “subject population” refers to at least five, preferably at least ten individual healthy human subjects.

Within the meaning of this invention, the term “geometric mean” refers to the mean of the log transformed data back-transformed to the original scale.

Within the meaning of this invention, the term “arithmetic mean” refers to the sum of all values of observation divided by the total number of observations.

Within the meaning of this invention, the parameter “AUC” corresponds to the area under the plasma concentration-time curve. The AUC value is proportional to the amount of active agent absorbed into the blood circulation in total and is hence a measure for the bioavailability.

Within the meaning of this invention, the parameter “AUCₜ₁₋ₜ₂” is provided in (ng/ml) hr and relates to the area under the plasma concentration-time curve from hour t1 to t2 and is calculated by the linear trapezoidal method, unless otherwise indicated. Other calculation methods are e.g. the logarithmic and linear log trapezoidal method.

Within the meaning of this invention, the parameter “Cₘₐₓ” is provided in (ng/ml) and relates to the maximum observed blood plasma concentration of the active agent.

Within the meaning of this invention, the parameter “Cₜ” is provided in (ng/ml) and relates to the blood plasma concentration of the active agent observed at hour t.

Within the meaning of this invention, the parameter “tₘₐₓ” is provided in hr and relates to the time point at which the Cₘₐₓ value is reached. In other words, tₘₐₓ is the time point of the maximum observed plasma concentration.

Within the meaning of this invention, the term “mean plasma concentration” is provided in (ng/ml) and is a mean of the individual plasma concentrations of active agent, e.g. nicotine, at each point in time.

Within the meaning of this invention, the term “coating composition” refers to a composition comprising all components of the matrix layer in a solvent, which may be coated onto the backing layer or release liner to form the matrix layer upon drying.

Within the meaning of this invention, the term “pressure sensitive adhesive composition” refers to a pressure sensitive adhesive at least in mixture with a solvent (e. g. n-heptane or ethyl acetate).

Within the meaning of this invention, the term “dissolve” refers to the process of obtaining a solution, which is clear and does not contain any particles, as visible to the naked eye.

Within the meaning of this invention, the term “solvent” refers to any liquid substance, which preferably is a volatile organic liquid such as methanol, ethanol, isopropanol, acetone, ethyl acetate, methylene chloride, hexane, n-heptane, toluene and mixtures thereof. Within the meaning of this invention, the term “hexane” refers to any hexane isomer and mixtures thereof, including pure n-hexane as well as a mixture with different hexane isomers and a large amount of n-hexane.

### BRIEF DESCRIPTION OF THE DRAWINGS

FIG. 1a depicts the cumulative permeated amount of nicotine for NicoDerm® CQ® as well as for a TTS prepared according to Ex. 1 for hours 0 to 32.

FIG. 1b depicts the nicotine skin permeation rate for NicoDerm® CQ® as well as for a TTS prepared according to Ex. 1 for hours 0 to 32.

FIG. 1c depicts the utilization of nicotine for NicoDerm® CQ® as well as for a TTS prepared according to Ex. 1 after 24 h.

FIG. 2a depicts the cumulative permeated amount of nicotine for Nicorette®, Nicotinell® as well as for TTS prepared according to Ex. 2a, 2b, 2c and 2d for hours 0 to 32.

FIG. 2b depicts the nicotine skin permeation rate for Nicorette®, Nicotinell® as well as for TTS prepared according to Ex. 2a, 2b, 2c and 2d for hours 0 to 32.

FIG. 2c depicts the utilization of nicotine for Nicorette®, Nicotinell® as well as for TTS prepared according to Ex. 2a, 2b, 2c and 2d after 24 h.

### DETAILED DESCRIPTION

### TTS Structure

The present invention is related to a transdermal therapeutic system for the transdermal administration of nicotine comprising a nicotine-containing layer structure.

The nicotine-containing layer structure in particular may contain a therapeutically effective amount of nicotine.

According to the present invention, the transdermal therapeutic system also comprises a silicone acrylic hybrid polymer, and the nicotine-containing layer structure comprises A) a backing layer and B) a nicotine-containing layer, and the nicotine-containing layer further comprises at least 0.8 mg/cm² nicotine.

Thus, in a first aspect, the present invention relates to a transdermal therapeutic system for the transdermal administration of nicotine comprising a nicotine-containing layer structure, said nicotine-containing layer structure comprising:
A) a backing layer; and
B) a nicotine-containing layer;
   wherein the transdermal therapeutic system comprises a silicone acrylic hybrid polymer, and wherein the nicotine-containing layer comprises at least 0.8 mg/cm² nicotine.

The backing layer is in particular substantially nicotine-impermeable.

Preferably, the nicotine-containing layer structure is a nicotine-containing self-adhesive layer structure, Thus, the transdermal therapeutic system for the transdermal administration of nicotine according to the present invention preferably comprises a nicotine-containing self-adhesive layer structure. The nicotine-containing self-adhesive layer structure may or may not comprise an additional skin contact layer. If the nicotine-containing layer itself is self-adhesive, an additional skin contact layer is not required and preferably is not present. The silicone acrylic hybrid polymer present in the transdermal therapeutic system is preferably present in the nicotine-containing self-adhesive layer structure and even more preferably in the nicotine-containing layer, and provides the self-adhesive properties.

The TTS according to the present invention may be a matrix-type TTS or a reservoir-type TTS. Preferably, the TTS according to the present invention is a matrix-type TTS.

In such a matrix-type TTS according to the invention, the nicotine is homogeneously dissolved and/or dispersed within a polymeric carrier, i.e. the matrix, which forms with the nicotine and optionally remaining ingredients a matrix layer. Thus, in such an embodiment, the nicotine-containing layer is a nicotine-containing matrix layer. The nicotine-containing layer structure may or may not comprise an additional skin contact layer. If the nicotine-containing matrix layer is self-adhesive, an additional skin contact layer is not required and preferably is not present. If a nicotine-containing matrix layer is prepared by laminating together two nicotine-containing matrix layers, which are of substantially the same composition, the resulting double layer is to be regarded as one nicotine-containing matrix layer.

In a reservoir-type TTS according to the present invention, a nicotine-containing reservoir is sealed between the backing layer and a rate-controlling membrane. Thus, the nicotine-containing layer is a nicotine-containing reservoir layer, which preferably comprises a liquid reservoir comprising the nicotine. The reservoir-type TTS typically additionally comprises a skin contact layer, wherein the reservoir layer and the skin contact layer are preferably separated by the rate-controlling membrane.

In specific embodiments, e. g. as outlined above, the nicotine-containing layer structure according to the present invention comprises or does not comprise an additional skin contact layer. The additional skin contact layer is preferably self-adhesive and provides for adhesion between the nicotine-containing layer structure and the skin of the patient during administration.

Without wishing to be bound to theory, it is believed that an additional skin contact layer underlying the nicotine-containing layer prevents a burst release of active in the first hours, e. g. in hours 0 to 8, and promotes maintaining sufficient active release over the whole duration of TTS application. Thus, in one preferred embodiment, the nicotine-containing self-adhesive layer structure comprises an additional skin contact layer, wherein the additional skin contact layer comprises or does not comprise nicotine, and preferably does not comprise nicotine. In this regard, it should be noted that the feature “does not comprise nicotine” is to be understood as meaning that the additional skin contact layer is manufactured such that it is nicotine-free. However, as outlined above, due to the concentration gradient, the nicotine may migrate from the nicotine-containing layer to the additional skin contact layer over time, until equilibrium is reached.

In preferred embodiments, the additional skin contact layer is a pressure-sensitive adhesive layer comprising a silicone acrylic hybrid polymer, a non-hybrid polymer and preferably a non-hybrid pressure-sensitive adhesive, or any mixture thereof. Most preferably, the additional skin contact layer is a pressure-sensitive adhesive layer comprising a silicone acrylic hybrid polymer.

Where the nicotine-containing layer structure comprises an additional skin contact layer, the nicotine-containing layer structure may or may not comprise a membrane which is located between the nicotine-containing layer and the additional skin contact layer, wherein the membrane is preferably a rate controlling membrane.

It is furthermore preferred that the nicotine-containing layer is directly attached to the backing layer, so that there is no additional layer between the backing layer and the nicotine-containing layer. Consequently, a layer structure of low complexity is obtained, which is advantageous, e. g., in terms of the costs for the manufacture.

In particular, it is preferred that the nicotine-containing layer structure comprises not more than 3 layers, and in one preferred embodiment, the nicotine-containing layer structure comprises 2 layers, i. e. only the backing layer and the nicotine-containing layer. Sufficient adhesion between the nicotine-containing layer structure and the skin of the patient during administration is then provided by the nicotine-containing layer, which is preferably a nicotine-containing pressure-sensitive adhesive layer.

The self-adhesive properties of the TTS according to the present invention are preferably provided by the silicone acrylic hybrid polymer, which is present in the TTS, preferably in the nicotine-containing layer structure, and more preferably in the nicotine-containing layer, which most preferable is a nicotine-containing matrix layer. Thus, in a preferred embodiment of the invention, the silicone acrylic hybrid polymer is a silicone acrylic hybrid pressure-sensitive adhesive. Further details regarding the silicone acrylic hybrid polymer according to the invention are provided further below.

According to certain embodiments of the invention, the TTS may further comprise an adhesive overlay. This adhesive overlay is in particular larger than the nicotine-containing layer structure and is attached thereto for enhancing the adhesive properties of the overall transdermal therapeutic system. Said adhesive overlay comprises also a backing layer and an adhesive layer. The area of said adhesive overlay adds to the overall size of the TTS but does not add to the area of release. The adhesive overlay comprises a self-adhesive polymer or a self-adhesive polymer mixture selected from the group of silicone acrylic hybrid polymers, acrylic polymers, polysiloxanes, polyisobutylenes, styrene-isoprene-styrene copolymers, and mixtures thereof, which may be identical to or different from any polymer or polymer mixture included in the nicotine-containing self-adhesive layer structure.

The nicotine-containing layer structure according to the invention, such as a nicotine-containing self-adhesive layer structure, is normally located on a detachable protective layer (release liner) from which it is removed immediately before application to the surface of the patient's skin. Thus, the TTS may further comprise a release liner. A TTS protected this way is usually stored in a blister pack or a seam-sealed pouch. The packaging may be child resistant and/or senior friendly.

### Nicotine-Containing Layer

As outlined in more detail above, the TTS according to a first aspect of the present invention comprises a nicotine-containing layer structure comprising a backing layer and a nicotine-containing layer, wherein the TTS comprises a silicone acrylic hybrid polymer.

In a preferred embodiment, the nicotine-containing layer comprises:
1. nicotine; and
2. a silicone acrylic hybrid polymer.

As outlined above, in one preferred embodiment of the invention, the nicotine-containing layer is a matrix layer, and preferably is a pressure-sensitive adhesive layer. In such a nicotine-containing matrix layer, the nicotine is homogeneously distributed within a polymer matrix. The polymer matrix preferably comprises the silicone acrylic hybrid polymer. Thus it is preferred according to the present invention that the nicotine-containing matrix layer comprises nicotine and the silicone acrylic hybrid polymer.

As outlined above, the nicotine-containing layer structure is preferably a nicotine-containing self-adhesive layer structure. Accordingly, it is preferred that the nicotine-containing layer is a nicotine-containing pressure-sensitive adhesive layer, and more preferably a nicotine-containing pressure-sensitive adhesive matrix layer.

In order to maintain a certain driving force and thus to achieve sufficient skin permeation, the nicotine amount and concentration in the nicotine-containing layer is preferably kept at a certain level. Thus, in preferred embodiments of the invention, the nicotine-containing layer comprises at least 0.90 mg/cm², preferably at least 0.95 mg/cm², more preferably at least 1.15 mg/cm² nicotine. On the other hand, if the concentration is too high, there is a risk that the nicotine is segregated from the nicotine-containing layer. Therefore, the nicotine-containing layer may in particular comprise less than 5.0 mg/cm², less than 4.0 mg/cm², less than 3.0 mg/cm² or less than 2.0 mg/cm² nicotine. In other preferred embodiments of the invention, the amount of nicotine in the nicotine-containing layer ranges from 2 to 15%, preferably from 3 to 12% and more preferably from 4 to 10% of the nicotine-containing layer.

In certain embodiments of the invention, the area weight of the nicotine-containing layer is at least 80 g/m² or is at least 90 g/m² or ranges from 80 to 300 g/m², preferably from 90 to 270 g/m², and more preferably from 100 to 230 g/m².

Without wishing to be bound by theory, it is believed that the advantageous features of the TTS according to the present invention, such as good in vitro skin permeation are inter alia achieved by the amount of nicotine contained in the TTS, which can be controlled two-way by adjusting concentration and/or the area weight of the nicotine-containing layers such as a nicotine-containing matrix layer.

As outlined above, the nicotine-containing layer preferably is a nicotine-containing pressure-sensitive adhesive layer comprising the silicone acrylic hybrid polymer. In such embodiments, the amount of the silicone acrylic hybrid polymer may in particular range from 55 to 98%, preferably from 70 to 97% or from 80 to 96% by weight based on the total weight of the nicotine-containing layer.

The nicotine-containing layer may also comprise a non-hybrid polymer, wherein the non-hybrid polymer preferably is a non-hybrid pressure-sensitive adhesive, wherein the non-hybrid polymer is preferably selected from polysiloxanes, polyisobutylenes, styrene-isoprene-styrene block copolymers and acrylic polymers. More details concerning the optional non-hybrid polymers are provided further below.

The nicotine-containing layer thus preferably is a nicotine-containing pressure-sensitive adhesive layer comprising the silicone acrylic hybrid polymer and/or a non-hybrid polymer, and the total polymer content, referring to the total amount of both silicone acrylic hybrid polymer and non-hybrid polymer, in particular may range from 75 to 98%, preferably from 80 to 98% and more preferably from 85 to 98% of the nicotine-containing layer. However, in certain embodiments, it is an advantage of the invention that only one matrix polymer is needed for the nicotine-containing layer. In fact, a sufficient level of nicotine concentration in the nicotine-containing layer can be achieved by using a silicone acrylic hybrid polymer as matrix polymer, even if a solvent with low boiling temperature/a volatile solvent, such as hexane, is used. Thus, preferably the nicotine-containing layer, which in particular is a nicotine-containing pressure-sensitive adhesive layer, comprises the silicone acrylic hybrid polymer but no non-hybrid polymer.

The nicotine-containing layer may also comprise further excipients or additives selected from the group consisting of cross-linking agents, solubilizers, fillers, tackifiers, film forming agents, plasticizers, stabilizers, softeners, substances for skincare, permeation enhancers, pH regulators, and preservatives. Details on such excipients and additives are provided further below.

As previously mentioned, the nicotine-containing layer provides the area of release. In preferred embodiments of the invention, the area of release ranges from 2 to 100 cm², preferably from 5 to 50 cm², and more preferably from 7 to 30 cm².

### Nicotine

The transdermal therapeutic system of the present invention comprises nicotine, and in particular therapeutically effective amounts of nicotine, in a nicotine-containing layer structure, i.e. in a nicotine-containing layer.

While the active agent may, in accordance with the present invention, be present in the TTS in protonated or in free base form, the free base form is preferred.

Thus, in certain embodiments, the nicotine in the nicotine-containing layer is included in the form of the free base.

In certain embodiments, the nicotine-containing layer is obtainable by incorporating the nicotine in the form of the free base. In a further embodiment, the nicotine-containing matrix layer is obtainable by incorporating the nicotine in the form of the free base.

In particular, at least 90 mol %, preferably at least 95 mol %, more preferably at least 98 mol % and most preferably at least 99 mol % of the nicotine in the nicotine-containing layer is present in the form of the free base.

The nicotine in the nicotine-containing layer may be completely dissolved, or the nicotine-containing layer may contain nicotine droplets, preferably constituted of nicotine free base.

The total amount of nicotine in the TTS is important for the amount of active released and also for the release rate. Thus, in certain preferred embodiments, the amount of nicotine contained in the TTS ranges from 5 to 90 mg, preferably from 8 to 75 mg, and most preferably from 10 to 60 mg.

### Silicone Acrylic Hybrid Polymer

The TTS according to the present invention comprises a silicone acrylic hybrid polymer. The silicone acrylic hybrid polymer comprises a polymerized hybrid species that includes silicone-based sub-species and acrylate-based sub-species that have been polymerized together. The silicone acrylic hybrid polymer thus comprises a silicone phase and an acrylic phase. Preferably, the silicone acrylic hybrid polymer is a silicone acrylic hybrid pressure-sensitive adhesive.

The silicone acrylic hybrid pressure-sensitive adhesives are usually supplied and used in solvents like n-heptane and ethyl acetate. The solids content of the pressure-sensitive adhesives is usually between 30% and 80%. The skilled person is aware that the solids content may be modified by adding a suitable amount of solvent.

Preferably, the weight ratio of silicone to acrylate in the silicone acrylic hybrid pressure-sensitive adhesive is from 5:95 to 95:5, or from 20:80 to 80:20, more preferably from 40:60 to 60:40, and most preferably the ratio of silicone to acrylate is about 50:50. Suitable silicone acrylic hybrid pressure-sensitive adhesives having a weight ratio of silicone to acrylate of 50:50 are, for example, the commercially available silicone acrylic hybrid pressure-sensitive adhesives 7-6102, Silicone/Acrylate Ratio 50/50, and 7-6302, Silicone/Acrylate Ratio 50/50, supplied in ethyl acetate by Dow Corning.

The preferred silicone acrylic hybrid pressure-sensitive adhesives in accordance with the invention are characterized by a solution viscosity at 25° C. and about 50% solids content in ethyl acetate of more than about 400 cP, or from about 500 cP to about 3,500 cP, in particular from about 1,000 cP to about 3,000 cP, more preferred from about 1,200 cP to about 1,800, or most preferred of about 1,500 cP or alternatively more preferred from about 2,200 cP to about 2,800 cP, or most preferred of about 2,500 cP, preferably as measured using a Brookfield RVT viscometer equipped with a spindle number 5 at 50 RPM.

These silicone acrylic hybrid pressure-sensitive adhesives may also be characterized by a complex viscosity at 0.1 rad/s at 30° C. of less than about 1.0e9 Poise, or from about 1.0e5 Poise to about 9.0e8 Poise, or more preferred from about 9.0e5 Poise to about 1.0e7 Poise, or most preferred about 4.0e6 Poise, or alternatively more preferred from about 2.0e6 Poise to about 9.0e7 Poise, or most preferred about 1.0e7 Poise, preferably as measured using a Rheometrics ARES rheometer, wherein the rheometer is equipped with 8 mm plates and the gap zeroed.

To prepare samples for measuring the rheological behavior using a Rheometrics ARES rheometer, between 2 and 3 grams of adhesive solution can be poured onto a SCOTCH-PAK 1022 fluoropolymer release liner and allow to sit for 60 minutes under ambient conditions. To achieve essentially solvent-free films of the adhesive, they can be placed in an oven at 110° C.+/−10° C. for 60 minutes. After removing from the oven and letting equilibrate to room temperature. the films can be removed from the release liner and folded over to form a square. To eliminate air bubbles the films can be compressed using a Carver press. The samples can then be loaded between the plates and are compressed to 1.5+/−0.1 mm at 30° C. The excess adhesive is trimmed and the final gap recorded. A frequency sweep between 0.01 to 100 rad/s can be performed with the following settings: Temperature=30° C.; strain=0.5-1% and data collected at 3 points/decade.

Suitable silicone acrylic hybrid pressure-sensitive adhesives which are commercially available include the PSA series 7-6100 and 7-6300 manufactured and supplied in n-heptane or ethyl acetate by Dow Corning (7-610X and 7-630X; X=1 n-heptane-based/X=2 ethyl acetate-based). For example, the 7-6102 silicone acrylic hybrid PSA having a silicone/acrylate ratio of 50/50 is characterized by a solution viscosity at 25° C. and about 50% solids content in ethyl acetate of 2,500 cP and a complex viscosity at 0.1 rad/s at 30° C. of 1.0e7 Poise. The 7-6302 silicone acrylic hybrid PSA having a silicone/acrylate ratio of 50/50 has a solution viscosity at 25° C. and about 50% solids content in ethyl acetate of 1,500 cP and a complex viscosity at 0.1 rad/s at 30° C. of 4.0e6 Poise.

Depending on the solvent in which the silicone acrylic hybrid pressure-sensitive adhesive is supplied, the arrangement of the silicone phase and the acrylic phase providing a silicone or acrylic continuous external phase and a corresponding discontinuous internal phase is different. If the silicone acrylic hybrid pressure-sensitive adhesive is provided in n-heptane, the composition contains a continuous, silicone external phase and a discontinuous, acrylic internal phase. If the silicone acrylic hybrid pressure-sensitive adhesive is provided in ethyl acetate, the composition contains a continuous, acrylic external phase and a discontinuous, silicone internal phase. After evaporating the solvent in which the silicone acrylic hybrid pressure-sensitive adhesive is provided, the phase arrangement of the resulting pressure-sensitive adhesive film or layer corresponds to the phase arrangement of the solvent-containing adhesive coating composition. For example, in the absence of any substance that may induce an inversion of the phase arrangement in a silicone acrylic hybrid pressure sensitive adhesive composition, a pressure-sensitive adhesive layer prepared from a silicone acrylic hybrid pressure-sensitive adhesive in n-heptane or hexane provides a continuous, silicone external phase and a discontinuous, acrylic internal phase, a pressure-sensitive adhesive layer prepared from a silicone acrylic hybrid pressure-sensitive adhesive in ethyl acetate provides a continuous, acrylic external phase and a discontinuous, silicone internal phase. In preferred embodiments, the nicotine-containing layer comprises 1. nicotine and 2. the silicone acrylic hybrid polymer, and is prepared using the silicone acrylic hybrid polymer in hexane. Therefore, the silicone acrylic hybrid polymer of the nicotine containing layer in such embodiments contains a continuous, silicone external phase and a discontinuous, acrylic internal phase. The phase arrangement of the compositions can, for example, be determined in peel force tests with pressure-sensitive adhesive films or layers prepared from the silicone acrylic hybrid PSA compositions which are attached to a siliconized release liner. The pressure-sensitive adhesive film contains a continuous, silicone external phase if the siliconized release liner cannot or can only hardly be removed from the pressure-sensitive adhesive film (laminated to a backing film) due to the blocking of the two silicone surfaces. Blocking results from the adherence of two silicone layers which comprise a similar surface energy. The silicone adhesive shows a good spreading on the siliconized liner and therefore can create a good adhesion to the liner. If the siliconized release liner can easily be removed the pressure-sensitive adhesive film contains a continuous, acrylic external phase. The acrylic adhesive has no good spreading due to the different surface energies and thus has a low or almost no adhesion to the siliconized liner.

According to a preferred embodiment of the invention the silicone acrylic hybrid polymer is a silicone acrylic hybrid pressure-sensitive adhesive obtainable from a silicon-containing pressure-sensitive adhesive composition comprising acrylate or methacrylate functionality. It is to be understood that the silicon-containing pressure-sensitive adhesive composition comprising acrylate or methacrylate functionality can include only acrylate functionality, only methacrylate functionality, or both acrylate functionality and methacrylate functionality.

According to certain embodiments of the invention the silicone acrylic hybrid pressure-sensitive adhesive comprises the reaction product of (a) a silicon-containing pressure-sensitive adhesive composition comprising acrylate or methacrylate functionality, (b) an ethylenically unsaturated monomer, and (c) an initiator. That is, the silicone acrylic hybrid pressure-sensitive adhesive is the product of the chemical reaction between these reactants ((a), (b), and (c)). In particular, the silicone acrylic hybrid pressure-sensitive adhesive includes the reaction product of (a) a silicon-containing pressure-sensitive adhesive composition comprising acrylate or methacrylate functionality, (b) a (meth)acrylate monomer, and (c) an initiator (i. e., in the presence of the initiator). That is, the silicone acrylic hybrid pressure-sensitive adhesive includes the product of the chemical reaction between these reactants ((a), (b), and (c)).

The reaction product of (a) a silicon-containing pressure-sensitive adhesive composition comprising acrylate or methacrylate functionality, (b) an ethylenically unsaturated monomer, and (c) an initiator may contain a continuous, silicone external phase and a discontinuous, acrylic internal phase or the reaction product of (a), (b), and (c) may contain a continuous, acrylic external phase and a discontinuous, silicone internal phase.

The silicon-containing pressure-sensitive adhesive composition comprising acrylate or methacrylate functionality (a) is typically present in the silicone acrylic hybrid pressure-sensitive adhesive in an amount of from 5 to 95, more typically 25 to 75, parts by weight based on 100 parts by weight of the hybrid pressure-sensitive adhesive.

The ethylenically unsaturated monomer (b) is typically present in the silicone acrylic hybrid pressure-sensitive adhesive in an amount of from 5 to 95, more typically 25 to 75, parts by weight based on 100 parts by weight of the hybrid pressure-sensitive adhesive.

The initiator (c) is typically present in the silicone acrylic hybrid pressure-sensitive adhesive in an amount of from 0.005 to 3, more typically from 0.01 to 2, parts by weight based on 100 parts by weight of the hybrid pressure-sensitive adhesive.

According to certain embodiments of the invention the silicon-containing pressure-sensitive adhesive composition comprising acrylate or methacrylate functionality (a) comprises the condensation reaction product of (a1) a silicone resin, (a2) a silicone polymer, and (a3) a silicon-containing capping agent which provides said acrylate or methacrylate functionality.

According to certain embodiments of the invention the silicon-containing pressure-sensitive adhesive composition comprising acrylate or methacrylate functionality (a) comprises the condensation reaction product of:
(a1) a silicone resin,
(a2) a silicone polymer, and
(a3) a silicon-containing capping agent which provides said acrylate or methacrylate functionality, wherein said silicon-containing capping agent is of the general formula XYR′_{b}SiZ_{3-b}, wherein
   X is a monovalent radical of the general formula AE—
      where E is —O— or —NH— and A is an acryl group or a methacryl group,
   Y is a divalent alkylene radical having from 1 to 6 carbon atoms,
   R′ is a methyl or a phenyl radical,
   Z is a monovalent hydrolyzable organic radical or a halogen, and
   b is 0 or 1;
wherein the silicone resin and silicone polymer are reacted to form a pressure-sensitive adhesive, wherein the silicon-containing capping agent is introduced prior to, during, or after the silicone resin and silicone polymer are reacted, and wherein:
   the silicon-containing capping agent reacts with the pressure-sensitive adhesive after the silicone resin and silicone polymer have been condensation reacted to form the pressure-sensitive adhesive; or
   the silicon-containing capping agent reacts in-situ with the silicone resin and silicone polymer.

According to certain embodiments of the invention the silicon-containing pressure-sensitive adhesive composition comprising acrylate or methacrylate functionality comprises the condensation reaction product of a pressure sensitive adhesive and a silicon-containing capping agent which provides said acrylate or methacrylate functionality. That is, the silicon-containing pressure sensitive adhesive composition comprising acrylate or methacrylate functionality is essentially a pressure sensitive adhesive that has been capped or end blocked with the silicon-containing capping agent which provides said acrylate or methacrylate functionality, wherein the pressure sensitive adhesive comprises the condensation reaction product of the silicone resin and the silicone polymer. Preferably, the silicone resin reacts in an amount of from 30 to 80 parts by weight to form the pressure sensitive adhesive, and the silicone polymer reacts in an amount of from 20 to 70 parts by weight to form the pressure sensitive adhesive. Both of these parts by weight are based on 100 parts by weight of the pressure sensitive adhesive. Although not required, the pressure sensitive adhesive may comprise a catalytic amount of a condensation catalyst. A wide array of silicone resins and silicone polymers are suitable to make up the pressure sensitive adhesive.

According to certain embodiments of the invention the silicone acrylic hybrid pressure-sensitive adhesive is the reaction product of:

(a) a silicon-containing pressure-sensitive adhesive composition comprising acrylate or methacrylate functionality that comprises the condensation reaction product of:
(a1) a silicone resin,
(a2) a silicone polymer, and
(a3) a silicon-containing capping agent which provides said acrylate or methacrylate functionality, wherein said silicon-containing capping agent is of the general formula XYR′_{b}SiZ_{3-b}, wherein
   X is a monovalent radical of the general formula AE—
      where E is —O— or —NH— and A is an acryl group or a methacryl group,
   Y is a divalent alkylene radical having from 1 to 6 carbon atoms,
   R′ is a methyl or a phenyl radical,
   Z is a monovalent hydrolyzable organic radical or a halogen, and
   b is 0 or 1;
      wherein the silicone resin and silicone polymer are reacted to form a pressure-sensitive adhesive, wherein the silicon-containing capping agent is introduced prior to, during, or after the silicone resin and silicone polymer are reacted, and wherein:
the silicon-containing capping agent reacts with the pressure-sensitive adhesive after the silicone resin and silicone polymer have been condensation reacted to form the pressure-sensitive adhesive; or
the silicon-containing capping agent reacts in-situ with the silicone resin and silicone polymer;
   (b) an ethylenically unsaturated monomer; and
   (c) an initiator.

The silicone acrylic hybrid composition used in the present invention may be described by being prepared by a method comprising the steps of:

(i) providing a silicon-containing pressure-sensitive adhesive composition comprising acrylate or methacrylate functionality that comprises the condensation reaction product of:
a silicone resin,
a silicone polymer, and
a silicon-containing capping agent which provides said acrylate or methacrylate functionality, wherein said silicon-containing capping agent is of the general formula XYR′_{b}SiZ_{3-b}, wherein
   X is a monovalent radical of the general formula AE—
      where E is —O— or —NH— and A is an acryl group or a methacryl group,
   Y is a divalent alkylene radical having from 1 to 6 carbon atoms,
   R′ is a methyl or a phenyl radical,
   Z is a monovalent hydrolyzable organic radical or a halogen, and
   b is 0 or 1;
wherein the silicone resin and silicone polymer are reacted to form a pressure-sensitive adhesive, wherein the silicon-containing capping agent is introduced prior to, during, or after the silicone resin and silicone polymer are reacted, and wherein:
   the silicon-containing capping agent reacts with the pressure-sensitive adhesive after the silicone resin and silicone polymer have been condensation reacted to form the pressure-sensitive adhesive; or
   the silicon-containing capping agent reacts in-situ with the silicone resin and silicone polymer;
      (ii) polymerizing an ethylenically unsaturated monomer and the silicon-containing pressure-sensitive adhesive composition comprising acrylate or methacrylate functionality of step (i) in the presence of an initiator to form a silicone acrylic hybrid composition, optionally at a temperature of from 50° C. to 100° C., or from 65° C. to 90° C.

During the polymerization of the ethylenically unsaturated monomer and the silicon-containing pressure-sensitive adhesive composition comprising acrylate or methacrylate functionality, the silicone to acrylic ratio can be controlled and optimized as desired. The silicone to acrylic ratio can be controlled by a wide variety of mechanisms in and during the method. An illustrative example of one such mechanism is the rate controlled addition of the ethylenically unsaturated monomer or monomers to the silicon-containing pressure-sensitive adhesive composition comprising acrylate or methacrylate functionality. In certain applications, it may be desirable to have the silicone-based sub-species, or the overall silicone content, to exceed the acrylate-based sub-species, or the overall acrylic content. In other applications, it may be desirable for the opposite to be true. Independent of the end application, it is generally preferred, as already described above, that the silicon-containing pressure-sensitive adhesive composition comprising acrylate or methacrylate functionality is preferably present in the silicone acrylic hybrid composition in an amount of from about 5 to about 95, more preferably from about 25 to about 75, and still more preferably from about 40 to about 60 parts by weight based on 100 parts by weight of the silicone acrylic hybrid composition.

According to a certain embodiment of the invention, the silicone acrylic hybrid composition used in the present invention may be described by being prepared by a method comprising the steps of:

(i) providing a silicon-containing pressure-sensitive adhesive composition comprising acrylate or methacrylate functionality that comprises the condensation reaction product of:
a silicone resin,
a silicone polymer, and
a silicon-containing capping agent which provides said acrylate or methacrylate functionality, wherein said silicon-containing capping agent is of the general formula XYR′_{b}SiZ_{3-b}, wherein
   X is a monovalent radical of the general formula AE—
      where E is —O— or —NH— and A is an acryl group or a methacryl group,
   Y is a divalent alkylene radical having from 1 to 6 carbon atoms,
   R′ is a methyl or a phenyl radical,
   Z is a monovalent hydrolyzable organic radical or a halogen, and
   b is 0 or 1;
wherein the silicone resin and silicone polymer are reacted to form a pressure-sensitive adhesive, wherein the silicon-containing capping agent is introduced prior to, during, or after the silicone resin and silicone polymer are reacted, and wherein:
   the silicon-containing capping agent reacts with the pressure-sensitive adhesive after the silicone resin and silicone polymer have been condensation reacted to form the pressure-sensitive adhesive; or
   the silicon-containing capping agent reacts in-situ with the silicone resin and silicone polymer;
      (ii) polymerizing an ethylenically unsaturated monomer and the silicon-containing pressure-sensitive adhesive composition comprising acrylate or methacrylate functionality of step (i) in a first solvent in the presence of an initiator at a temperature of from 50° C. to 100° C. to form a silicone acrylic hybrid composition;
      (iii) removing the first solvent; and
      (iv) adding a second solvent to form the silicone acrylic hybrid composition, wherein the phase arrangement of the silicone acrylic hybrid composition is selectively controlled by selection of the second solvent.

The silicone acrylic hybrid PSA composition used in the present invention may also be described by being prepared by a method comprising the steps of:

(i) providing a silicon-containing pressure-sensitive adhesive composition comprising acrylate or methacrylate functionality that comprises the condensation reaction product of:
a silicone resin,
a silicone polymer, and
a silicon-containing capping agent which provides said acrylate or methacrylate functionality, wherein said silicon-containing capping agent is of the general formula XYR′_{b}SiZ_{3-b}, wherein
   X is a monovalent radical of the general formula AE—
      where E is —O— or —NH— and A is an acryl group or a methacryl group,
   Y is a divalent alkylene radical having from 1 to 6 carbon atoms,
   R′ is a methyl or a phenyl radical,
   Z is a monovalent hydrolyzable organic radical or a halogen, and
   b is 0 or 1;
wherein the silicone resin and silicone polymer are reacted to form a pressure-sensitive adhesive, wherein the silicon-containing capping agent is introduced prior to, during, or after the silicone resin and silicone polymer are reacted, and wherein:
   the silicon-containing capping agent reacts with the pressure-sensitive adhesive after the silicone resin and silicone polymer have been condensation reacted to form the pressure-sensitive adhesive; or
   the silicon-containing capping agent reacts in-situ with the silicone resin and silicone polymer;
      (ii) polymerizing an ethylenically unsaturated monomer and the silicon-containing pressure-sensitive adhesive composition comprising acrylate or methacrylate functionality of step (i) in a first solvent in the presence of an initiator at a temperature of from 50° C. to 100° C. to form a silicone acrylic hybrid composition;
      (iii) adding a processing solvent, wherein the processing solvent has a higher boiling point than the first solvent, and
      (iv) applying heat at a temperature of from 70° C. to 150° C. such that a majority of the first solvent is selectively removed;
      (v) removing the processing solvent; and
      (vi) adding a second solvent to form the silicone acrylic hybrid composition, wherein the phase arrangement of the silicone acrylic hybrid composition is selectively controlled by selection of the second solvent.

The silicone resin according to the previous paragraphs may contain a copolymer comprising triorganosiloxy units of the formula R^{X}₃SiO_{1/2} and tetrafunctional siloxy units of the formula SiO_{4/2} in a ratio of from 0.1 to 0.9, preferably of about 0.6 to 0.9, triorganosiloxy units for each tetrafunctional siloxy unit. Preferably, each R^{X} independently denotes a monovalent hydrocarbon radical having from 1 to 6 carbon atoms, vinyl, hydroxyl or phenyl groups.

The silicone polymer according to the previous paragraphs may comprise at least one polydiorganosiloxane and is preferably end-capped (end-blocked) with a functional group selected from the group consisting of hydroxyl groups, alkoxy groups, hydride groups, vinyl groups, or mixtures thereof. The diorganosubstituent may be selected from the group consisting of dimethyl, methylvinyl, methylphenyl, diphenyl, methylethyl, (3,3,3-trifluoropropyl)methyl and mixtures thereof. Preferably, the diorganosubstituents contain only methyl groups. The molecular weight of polydiorganosiloxane will typically range from about 50,000 to about 1,000,000, preferably, from about 80,000 to about 300,000. Preferably, the polydiorganosiloxane comprises AR^{X}SiO units terminated with endblocking TR^{X}ASiO_{1/2} units, wherein the polydiorganosiloxane has a viscosity of from about 100 centipoise to about 30,000,000 centipoise at 25° C., each A radical is independently selected from R^{X} or halohydrocarbon radicals having from 1 to 6 carbon atoms, each T radical is independently selected from the group consisting of R^{X}, OH, H or OR^{Y}, and each R^{Y} is independently an alkyl radical having from 1 to 4 carbon atoms.

As an example using forms of the preferred silicone resin and the preferred silicone polymer, one type of pressure sensitive adhesive is made by:

mixing (i) from 30 to 80 inclusive parts by weight of at least one resin copolymer containing silicon-bonded hydroxyl radicals and consisting essentially of R^{X}₃SiO_{1/2} units and SiO_{4/2} units in a mole ratio of 0.6 to 0.9 R^{X}₃SiO_{1/2} units for each SiO_{4/2} unit present, (ii) between about 20 and about 70 parts by weight of at least one polydiorganosiloxane comprising AR^{X}SiO units terminated with endblocking TR^{X}ASiO_{1/2} units, wherein the polydiorganosiloxane has a viscosity of from about 100 centipoise to about 30,000,000 centipoise at 25° C. and each R^{X} is a monovalent organic radical selected from the group consisting of hydrocarbon radicals of from 1 to 6 inclusive carbon atoms, each A radical is independently selected from R^{X} or halohydrocarbon radicals having from 1 to 6 inclusive carbon atoms, each T radical is independently selected from the group consisting of R^{X}, OH, H or OR^{Y}, and each R^{Y} is independently an alkyl radical of from 1 to 4 inclusive carbon atoms; a sufficient amount of (iii) at least one of the silicon-containing capping agents, also referred to throughout as endblocking agents, described below and capable of providing a silanol content, or concentration, in the range of 5,000 to 15,000, more typically 8,000 to 13,000, ppm, when desirable an additional catalytic amount of (iv) a mild silanol condensation catalyst in the event that none is provided by (ii), and when necessary, an effective amount of (v) an organic solvent which is inert with respect to (i), (ii), (iii) and (iv) to reduce the viscosity of a mixture of (i), (ii), (iii), and (iv), and condensing the mixture of (i), (ii), (iii) and (iv) at least until a substantial amount of the silicon-containing capping agent or agents have reacted with the silicon-bonded hydroxyl radicals and T radicals of (i) and (ii). Additional organosilicon endblocking agents can be used in conjunction with the silicon-containing capping agent or agents (iii) of the present invention.

The silicon-containing capping agent according to the previous paragraphs may be selected from the group of acrylate functional silanes, acrylate functional silazanes, acrylate functional disilazanes, acrylate functional disiloxanes, methacrylate functional silanes, methacrylate functional silazanes, methacrylate functional disilazanes, meth-acrylate functional disiloxanes, and combinations thereof and may be described as to be of the general formula XYR′_{b}SiZ_{3-b}, wherein X is a monovalent radical of the general formula AE— where E is —O— or —NH— and A is an acryl group or a methacryl group, Y is a divalent alkylene radical having from 1 to 6 carbon atoms, R′ is a methyl or a phenyl radical, Z is a monovalent hydrolyzable organic radical or a halogen, and b is 0, 1 or 2. Preferably, the monovalent hydrolyzable organic radical is of the general formula R″0—where R″ is an alkylene radical. Most preferably, this particular endblocking agent is selected from the group of 3-methacryloxypropyldimethylchlorosilane, 3-methacryloxypropyldichlorosilane, 3-methacryloxypropyltrichlorosilane, 3-methacryloxypropyldimethylmethoxysilane, 3-methacryloxypropylmethyldimethoxysilane, 3-meth-acryloxypropyltrimethoxysilane, 3-methacryloxypropyldimethylethoxysilane, 3-methacryloxypropylmethyldiethoxysilane, 3-methacryloxypropyltriethoxysilane, (methacryloxymethyl)dimethylmethoxysilane, (methacryloxymethyl)methyldimethoxysilane, (methacryloxymethyl)trimethoxysilane, (methacryloxymethyl)dimethylethoxysilane, (methacryloxymethyl)methyldiethoxysilane, methacryloxymethyltriethoxysilane, methacryloxy-propyltriisopropoxysilane, 3-methacryloxypropyldimethylsilazane, 3-acryloxy-propyldimethylchlorosilane, 3-acryloxypropyldichlorosilane, 3-acryloxypropyl-trichlorosilane, 3-acryloxypropyldimethylmethoxysilane, 3-acryloxy-propylmethyldimethoxysilane, 3-acryloxypropyltrimethoxysilane, 3-acryloxypropyl-dimethylsilazane, and combinations thereof.

The ethylenically unsaturated monomer according to the previous paragraphs can be any monomer having at least one carbon-carbon double bond. Preferably, the ethylenically unsaturated monomer according to the previous paragraphs may be a compound selected from the group consisting of aliphatic acrylates, aliphatic methacrylates, cycloaliphatic acrylates, cycloaliphatic methacrylates, and combinations thereof. It is to be understood that each of the compounds, the aliphatic acrylates, the aliphatic methacrylates, the cycloaliphatic acrylates, and the cycloaliphatic methacrylates, include an alkyl radical. The alkyl radicals of these compounds can include up to 20 carbon atoms. The aliphatic acrylates that may be selected as one of the ethylenically unsaturated monomers are selected from the group consisting of methyl acrylate, ethyl acrylate, propyl acrylate, n-butyl acrylate, iso-butyl acrylate, tert-butyl acrylate, hexyl acrylate, 2-ethylhexyl acrylate, iso-octyl acrylate, iso-nonyl acrylate, iso-pentyl acrylate, tridecyl acrylate, stearyl acrylate, lauryl acrylate, and mixtures thereof. The aliphatic methacrylates that may be selected as one of the ethylenically unsaturated monomers are selected from the group consisting of methyl methacrylate, ethyl methacrylate, propyl methacrylate, n-butyl methacrylate, iso-butyl meth-acrylate, tert-butyl methacrylate, hexyl methacrylate, 2-ethylhexyl methacrylate, iso-octyl methacrylate, iso-nonyl methacrylate, iso-pentyl methacrylate, tridecyl methacrylate, stearyl methacrylate, lauryl methacrylate, and mixtures thereof. The cycloaliphatic acrylate that may be selected as one of the ethylenically unsaturated monomers is cyclohexyl acrylate, and the cycloaliphatic methacrylate that may be selected as one of the ethylenically unsaturated monomers is cyclohexyl methacrylate.

It is to be understood that the ethylenically unsaturated monomer used for preparing the silicone acrylic hybrid pressure sensitive adhesive may be more than one ethylenically unsaturated monomer. That is, a combination of ethylenically unsaturated monomers may be polymerized, more specifically co-polymerized, along with the silicon-containing pressure sensitive adhesive composition comprising acrylate or methacrylate functionality and the initiator. According to a certain embodiment of the invention, the silicone acrylic hybrid pressure-sensitive adhesive is prepared by using at least two different ethylenically unsaturated monomers, preferably selected from the group of 2-ethylhexyl acrylate and methyl acrylate, more preferably in a ratio of 50% 2-ethylhexyl acrylate and 50% methyl acrylate, or in a ratio of 60% 2-ethylhexyl acrylate and 40% methyl acrylate as the acrylic monomer.

The initiator according to the previous paragraphs may be any substance that is suitable to initiate the polymerization of the silicon-containing pressure sensitive adhesive composition comprising acrylate or methacrylate functionality and the ethylenically unsaturated monomer to form the silicone acrylic hybrid. For example, free radical initiators selected from the group of peroxides, azo compounds, redox initiators, and photo-initiators may be used.

Further suitable silicone resins, silicone polymers, silicon-containing capping agents, ethylenically unsaturated monomers, and initiators that can be used in accordance with the previous paragraphs are detailed in WO 2007/145996, EP 2 599 847 A1, and WO 2016/130408.

According to a certain embodiment of the invention, the silicone acrylic hybrid polymer comprises a reaction product of a silicone polymer, a silicone resin and an acrylic polymer, wherein the acrylic polymer is covalently self-crosslinked and covalently bound to the silicone polymer and/or the silicone resin.

According to a certain other embodiment of the invention, the silicone acrylic hybrid polymer comprises a reaction product of a silicone polymer, a silicone resin and an acrylic polymer, wherein the silicone resin contains triorganosiloxy units R₃SiO_{1/2} where R is an organic group, and tetrafunctional siloxy units SiO_{4/2} in a mole ratio of from 0.1 to 0.9 R₃SiO_{1/2} units for each SiO_{4/2}.

The acrylic polymer may comprise at least an alkoxysilyl functional monomer, polysiloxane-containing monomer, halosilyl functional monomer or alkoxy halosilyl functional monomer. Preferably, the acrylic polymer is prepared from alkoxysilyl functional monomers selected from the group consisting of trialkoxylsilyl (meth)acrylates, dialkoxyalkylsilyl (meth)acrylates, and mixtures thereof, or comprises end-capped alkoxysilyl functional groups. The alkoxysilyl functional groups may preferably be selected from the group consisting of trimethoxylsilyl groups, dimethoxymethylsilyl groups, triethoxylsilyl, diethoxymethylsilyl groups and mixtures thereof.

The acrylic polymer may also be prepared from a mixture comprising polysiloxane-containing monomers, preferably from a mixture comprising polydimethylsiloxane mono (meth)acrylate.

The silyl functional monomers will typically be used in amounts of from 0.2 to 20 weight percent of the acrylic polymer, more preferably the amount of silyl functional monomers will range from about 1.5 to about 5 weight percent of the acrylic polymer.

The amount of polysiloxane-containing monomer will typically be used in amounts of from 1.5 to 50 weight percent of the acrylic polymer, more preferably the amount of polysiloxane-containing monomers will range from 5 to 15 weight percent of the acrylic polymer.

Alternatively, the acrylic polymer comprises a block or grafted copolymer of acrylic and polysiloxane. An example of a polysiloxane block copolymer is polydimethylsiloxane-acrylic block copolymer. The preferred amount of siloxane block is 10 to 50 weight percent of the whole block polymer.

The acrylic polymer comprises alkyl (meth)acrylate monomers. Preferred alkyl (meth)acrylates which may be used have up to about 18 carbon atoms in the alkyl group, preferably from 1 to about 12 carbon atoms in the alkyl group. Preferred low glass transition temperature (Tg) alkyl acrylate with a homopolymer Tg of less than about 0° C. have from about 4 to about 10 carbon atoms in the alkyl group and include butyl acrylate, amyl acrylate, hexyl acrylate, 2-ethylhexyl acrylate, octyl acrylate, isooctyl acrylate, decyl acrylate, isomers thereof, and combinations thereof. Particularly preferred are butyl acrylate, 2-ethylhexyl acrylate and isooctyl acrylate. The acrylic polymer components may further comprise (meth)acrylate monomers having a high Tg such as methyl acrylate, ethyl acrylate, methyl methacrylate and isobutyl methacrylate.

The acrylic polymer component may further comprise a polyisobutylene group to improve cold flow properties of the resultant adhesive.

The acrylic polymer components may comprise nitrogen-containing polar monomers. Examples include N-vinyl pyrrolidone, N-vinyl caprolactam, N-tertiary octyl acrylamide, dimethyl acrylamide, diacetone acrylamide, N-tertiary butyl acrylamide, N-isopropyl acrylamide, cyanoethylacrylate, N-vinyl acetamide and N-vinyl formamide.

The acrylic polymer component may comprise one or more hydroxyl containing monomers such as 2-hydroxyethyl acrylate, 2-hydroxyethyl methacrylate, hydroxypropyl acrylate and/or hydroxypropyl methacrylate.

The acrylic polymer components may, if desired, comprise carboxylic acid containing monomers. Useful carboxylic acids preferably contain from about 3 to about 6 carbon atoms and include, among others, acrylic acid, methacrylic acid, itaconic acid, β-carboxyethyl acrylate and the like. Acrylic acid is particularly preferred.

Other useful, well known co-monomers include vinyl acetate, styrene, cyclohexyl acrylate, alkyl di(meth)acrylates, glycidyl methacrylate and allyl glycidyl ether, as well as macromers such as, for example, poly(styryl)methacrylate.

One acrylic polymer component that can be used in the practice of the invention is an acrylic polymer that comprises from about 90 to about 99.5 wt % of butyl acrylate and from about 0.5 to about 10 wt % dimethoxymethylsilyl methacrylate.

According to a certain embodiment of the invention the silicone acrylic hybrid polymer may be prepared by a) reacting silicone polymer with silicone resin to form a resultant product, b) reacting the resultant product of a) with an acrylic polymer containing reactive functionality, wherein the components are reacted in an organic solvent.

According to a certain embodiment of the invention the silicone acrylic hybrid polymer may be prepared by a) reacting a silicone resin with an acrylic polymer containing reactive functionality to for ii a resultant product, b) reacting the resultant product of a) with silicone polymer, wherein the components are reacted in an organic solvent.

According to a certain embodiment of the invention the silicone acrylic hybrid polymer may be prepared by a) reacting a silicone polymer with an acrylic polymer containing reactive functionality to form a resultant product, b) reacting the resultant product of a) with silicone resin, wherein the components are reacted in an organic solvent.

Further suitable acrylic polymers, silicone resins, and silicone polymers that can be used for chemically reacting together a silicone polymer, a silicone resin and an acrylic polymer to provide a silicone acrylic hybrid polymer in accordance with the previous paragraphs are detailed in WO 2010/124187.

According to certain embodiments of the invention, the silicone acrylic hybrid polymer used in the TTS is blended with one or more non-hybrid polymers, preferably the silicone acrylic hybrid polymer is blended with one or more non-hybrid pressure sensitive adhesives (e. g. pressure-sensitive adhesives based on polysiloxane or acrylates).

### Non-Hybrid Polymers

According to a certain embodiment of the invention, the TTS comprises one or more non-hybrid polymers (e.g. non-hybrid pressure-sensitive adhesives) in addition to the silicone acrylic hybrid polymer. Non-hybrid polymers (e.g. non-hybrid pressure-sensitive adhesives) are polymers (e.g. polymer-based pressure-sensitive adhesives) which do not include a hybrid species. Preferred are non-hybrid polymers (e.g. non-hybrid pressure-sensitive adhesives) based on polysiloxanes, acrylates, polyisobutylenes, or styrene-isoprene-styrene block copolymers.

The non-hybrid polymers (e. g. the non-hybrid pressure-sensitive adhesives) may be contained in the active agent-containing layer structure and/or in the adhesive overlay.

Non-hybrid pressure-sensitive adhesives are usually supplied and used in solvents like n-heptane and ethyl acetate. The solids content of the pressure-sensitive adhesives is usually between 30% and 80%.

Suitable non-hybrid polymers according to the invention are commercially available e.g. under the brand names Bio-PSAs (polysiloxanes), Oppanol™ (polyisobutylenes), JSR-SIS (a styrene-isoprene-styrene copolymer) or Dura-Tak™ (acrylic polymers).

Pressure-sensitive adhesives based on polysiloxanes may also be referred to as silicone-based pressure-sensitive adhesives, or silicone pressure-sensitive adhesives. Pressure-sensitive adhesives based on polysiloxanes may have a solids content preferably between 60% and 80%. Such silicone-based PSAs need, unlike other organic pressure sensitive adhesives, no additives like antioxidants, stabilizers, plasticizers, catalysts or other potentially extractable ingredients. These pressure-sensitive adhesives provide for suitable tack and for quick bonding to various skin types, including wet skin, suitable adhesive and cohesive qualities, long lasting adhesion to the skin, a high degree of flexibility, a peiineability to moisture, and compatibility to many actives and film-substrates. It is possible to provide them with sufficient amine resistance and therefore enhanced stability in the presence of amines. Such pressure-sensitive adhesives are based on a resin-in-polymer concept wherein, by condensation reaction of silanol end blocked polydimethylsiloxane with a silica resin, a polysiloxane is prepared which for amine stability the residual silanol functionality is additionally capped with trimethylsiloxy groups. The silanol end blocked polydimethylsiloxane content contributes to the viscous component of the visco-elastic behavior, and impacts the wetting and the spreadability properties of the adhesive. The resin acts as a tackifying and reinforcing agent, and participates in the elastic component. The correct balance between silanol end blocked polydimethylsiloxane and resin provides for the correct adhesive properties.

Examples of silicone-based PSA compositions which are commercially available include the standard BIO-PSA series (7-4400, 7-4500 and 7-4600 series) and the amine compatible (endcapped) BIO-PSA series (7-4100, 7-4200 and 7-4300 series), manufactured and typically supplied in n-heptane or ethyl acetate by Dow Corning. For example, BIO-PSA 7-4201 is characterized by a solution viscosity at 25° C. and about 60% solids content in heptane of 450 mPa s and a complex viscosity at 0.01 rad/s at 30° C. of 1×10⁸ Poise. BIO-PSA 7-4301 has a solution viscosity at 25° C. and about 60% solids content in heptane of 500 mPa s and a complex viscosity at 0.01 rad/s at 30° C. of 5×10⁶ Poise.

The polysiloxanes are supplied and used in solvents like n-heptane, ethyl acetate or other volatile silicone fluids. The solids content of polysiloxanes in solvents is usually between 60 and 85%, preferably between 70 and 80%. The skilled person is aware that the solids content may be modified by adding a suitable amount of solvent.

The preferred pressure-sensitive adhesives based on polysiloxanes in accordance with the invention are characterized by a solution viscosity at 25° C. and 60% solids content in n-heptane of more than about 150 mPa s, or from about 200 mPa s to about 700 mPa s, preferably as measured using a Brookfield RVT viscometer equipped with a spindle number 5 at 50 rpm. These may also be characterized by a complex viscosity at 0.01 rad/s at 30° C. of less than about 1×10⁹ Poise or from about 1×10⁵ to about 9×10⁸ Poise.

Suitable polyisobutylenes according to the invention are available under the tradename Oppanol®. Combinations of high-molecular weight polyisobutylenes (B100/B80) and low-molecular weight polyisobutylenes (B10, B11, B12, B13) may be used. Suitable ratios of low-molecular weight polyisobutylene to high-molecular weight polyisobutylene are in the range of from 100:1 to 1:100, preferably from 95:5 to 40:60, more preferably from 90:10 to 80:20. A preferred example for a polyisobutylene combination is B10/B100 in a ratio of 85/15. Oppanol® B100 has a viscosity average molecular weight of 1,110,000, and a weight average molecular weight M_{w} of 1,550,000, and an average molecular weight distribution M_{w}/Mₙ of 2.9. Oppanol® B10 has a viscosity average molecular weight Mᵥ of 40,000, and a weight average molecular weight M_{w} of 53,000, and an average molecular weight distribution M_{w}/Mₙ of 3.2. In certain embodiments, polybutene may be added to the polyisobutylenes. The solids content of polyisobutylenes in solvents is usually between 30 and 50%, preferably between 35 and 40%. The skilled person is aware that the solids content may be modified by adding a suitable amount of solvent.

In a preferred embodiment, the non-hybrid polymer is selected from acrylic polymers. Preferably, the acrylic polymers are pressure-sensitive adhesives based on acrylates and may also be referred to as acrylate-based pressure-sensitive adhesives, or acrylate pressure-sensitive adhesives. Pressure-sensitive adhesives based on acrylates may have a solids content preferably between 30% and 60%. Acrylic polymers, and in particular acrylate-based pressure-sensitive adhesives may or may not comprise functional groups such as hydroxy groups, carboxylic acid groups, neutralized carboxylic acid groups and mixtures thereof. Thus, the term “functional groups” in particular refers to hydroxy- and carboxylic acid groups, and deprotonated carboxylic acid groups.

Corresponding commercial products are available e.g. from Henkel under the tradename Duro Tak®. Such acrylate-based pressure-sensitive adhesives are based on monomers selected from one or more of acrylic acid, butylacrylate, 2-ethylhexylacrylate, glycidylmethacrylate, 2-hydroxyethylacrylate, methylacrylate, methylmethacrylate, t-octylacrylamide and vinylacetate, and are provided in ethyl acetate, heptanes, n-heptane, hexane, methanol, ethanol, isopropanol, 2,4-pentanedione, toluene or xylene or mixtures thereof.

Specific acrylate-based pressure-sensitive adhesives are available as:
Duro-Tak™ 387-2287 or Duro-Tak™ 87-2287 (a copolymer based on vinyl acetate, 2-ethylhexyl-acrylate, 2-hydroxyethyl-acrylate and glycidyl-methacrylate provided as a solution in ethyl acetate without cross-linking agent),
Duro-Tak™ 387-2516 or Duro-Tak™ 87-2516 (a copolymer based on vinyl acetate, 2-ethylhexyl-acrylate, 2-hydroxyethyl-acrylate and glycidyl-methacrylate provided as a solution in ethyl acetate, ethanol, n-heptane and methanol with a titanium cross-linking agent),
Duro-Tak™ 387-2051 or Duro-Tak™ 87-2051 (a copolymer based on acrylic acid, butylacrylate, 2-ethylhexylacrylate and vinyl acetate, provided as a solution in ethyl acetate and heptane),
Duro-Tak™ 387-2353 or Duro-Tak™ 87-2353 (a copolymer based on acrylic acid, 2-ethylhexylacrylate, glycidylmethacrylate and methylacrylate, provided as a solution in ethyl acetate and hexane),
Duro-Tak™ 87-4098 (a copolymer based on 2-ethylhexyl-acrylate and vinyl acetate, provided as a solution in ethyl acetate).

Additional polymers may also be added to enhance cohesion and/or adhesion.

Certain polymers in particular reduce the cold flow and are thus in particular suitable as additional polymer. A polymeric matrix may show a cold flow, since such polymer compositions often exhibit, despite a very high viscosity, the ability to flow very slowly. Thus, during storage, the matrix may flow to a certain extent over the edges of the backing layer. This is a problem with storage stability and can be prevented by the addition of certain polymers. A basic acrylate polymer (e. g. Eudragit® E100) may e.g. be used to reduce the cold flow. Thus, in certain embodiments, the matrix layer composition comprises additionally a basic polymer, in particular an amine-functional acrylate as e.g. Eudragit® E100. Eudragit® E100 is a cationic copolymer based on dimethylaminoethyl methacrylate, butyl methacrylate, and methyl methacrylate with a ratio of 2:1:1. The monomers are randomly distributed along the copolymer chain. Based on SEC method, the weight average molar mass (Mw) of Eudragit® E100 is approximately 47,000 g/mol.

Adhesion force tests were performed with the nicotine-containing adhesive matrix layer using a tensile strength testing machine. Prior testing the samples were equilibrated 24 hours under controlled conditions at approx. room temperature (23±2° C.) and approx. 50% rh (relative humidity). Further, the samples were cut into pieces with a fixed width of 25 mm and a suitable length. The first millimeters of the abhesively equipped foil was pulled off and a splicing tape is applied to the opened adhesive side. Then, the abhesively foil was totally removed and the sample was placed with the adhesive surface in longitudinal direction onto the center of the cleaned testing plate (aluminum or staninless steel). The testing plate was fixed to the lower clamp of the tensile strength machine. The machine was adjusted to zero, the splicing tape was gripped into the upper clamp of the machine. The pull angle was set to 90°. After measurement of the adhesion force of three samples, the mean value of the adhesion force was calculated. The measurement value is based on units “N/sample width” [N/25 mm].

Tack (the force which is required to separate an object from an adhesive surface after a short time of contact) tests were performed with the nicotine-containing adhesive matrix layer in accordance with the Standard Test Method for Pressure-Sensitive Tack of Adhesives Using an Inverted Probe Machine (ASTM D 2979-01; Reapproved 2009) using a probe tack tester PT-1000 (ChemInstruments). Prior to testing the samples were equilibrated 24 hours under controlled conditions at approx. room temperature (23±2° C.) and approx. 50% rh. For determining the tack, the tip of a cleaned probe with a diameter of 5 mm was brought into contact with the adhesive surface of the nicotine-containing adhesive matrix layer for 1 second, at a defined rate (10±0.1 mm/s), under defined pressure (9.79±0.10 kPa), at a given temperature (23±2° C.) and the bond formed between probe and the adhesive was subsequently broken at the same rate. Tack was measured as the maximum force required, to break the adhesion bond (see ASTM D2979-01; Reapproved 2009). After finalization the mean value from the individual results of three associated samples were calculated and the mean tack value reported in [N].

### Further Additives

The TTS according to the invention, and in particular the nicotine-containing layer may further comprise at least one excipient or additive. In particular, the nicotine-containing layer comprises further excipients or additives selected from the group consisting of cross-linking agents, solubilizers, fillers, tackifiers, film-forming agents, plasticizers, stabilizers, softeners, substances for skincare, permeation enhancers, pH regulators, and preservatives. Such additives may be present in the nicotine-containing layer in an amount of from 0.001 to 10% by weight.

It should be noted that in pharmaceutical formulations, the formulation components are categorized according to their physicochemical and physiological properties, and in accordance with their function. This means in particular that a substance or a compound falling into one category is not excluded from falling into another category of formulation component. E. g. a certain polymer can be a non-hybrid polymer but also a film-forming agent. Some substances may e.g. be a typical softener but at the same time act as a permeation enhancer. The skilled person is able to determine based on his general knowledge in which category or categories of formulation component a certain substance or compound belongs to. In the following, details on the excipients and additives are provided which are, however, not to be understood as being exclusive. Other substances not explicitly listed in the present description may be as well used in accordance with the present invention, and substances and/or compounds explicitly listed for one category of formulation component are not excluded from being used as another formulation component in the sense of the present invention.

The cross-linking agent in particular may be selected from the group consisting of aluminium and titanium cross-linking agents such as aluminium acetylacetonate, titanium acetylacetonate or polybutyltitanate, and preferably is a titanium cross-linking agent. The amount of cross-linking agent may range from 0.005 to 1%, and preferably from 0.01 to 0.1% of the nicotine-containing layer. The nicotine-containing layer may also comprise a polymer which is self-crosslinking, i.e. comprises a cross-linking functional group such as glycidyl groups, which reacts upon heating. According to a further specific embodiment, the nicotine-containing layer comprises a cross-linking agent as above and a self-crosslinking polymer.

In one embodiment, the nicotine-containing layer further comprises a stabilizer, wherein the stabilizer is preferably selected from tocopherol and ester derivatives thereof and ascorbic acid and ester derivatives thereof. Preferred stabilizers include tocopherol and ester derivatives thereof, ascorbic acid and ester derivatives thereof, butyl hydroxyl anisol and butyl hydroxyl-toluene. Particularly preferred is tocopherol.

In case the nicotine-containing layer is required to have self-adhesive properties and one or more polymers is/are selected which does/do not provide sufficient self-adhesive properties, a tackifier is added. The tackifier may be selected from triglycerides, dipropylene glycol, resins, resin esters, terpenes and derivatives thereof, ethylene vinyl acetate adhesives, dimethylpolysiloxanes and polybutenes.

The nicotine-containing layer may comprise a permeation enhancer. Permeation enhancers are substances, which influence the barrier properties of the stratum corneum in the sense of increasing the active agent permeability. Some examples of permeation enhancers are polyhydric alcohols such as dipropylene glycol, propylene glycol, and polyethylene glycol; oils such as olive oil, squalene, and lanolin; fatty ethers such as cetyl ether and oleyl ether; fatty acid esters such as isopropyl myristate; urea and urea derivatives such as allantoin; polar solvents such as dimethyldecylphosphoxide, methylcetylsulfoxide, dimethylaurylamine, dodecyl pyrrolidone, isosorbitol, dimethylacetonide, dimethylsulfoxide, decylmethylsulfoxide, and dimethylformamide; salicylic acid; amino acids; benzyl nicotinate; and higher molecular weight aliphatic surfactants such as lauryl sulfate salts. Other agents include oleic and linoleic acids, ascorbic acid, panthenol, butylated hydroxytoluene, tocopherol, tocopheryl acetate, tocopheryl linoleate, propyl oleate, and isopropyl palmitate. Preferably, the permeation enhancer is selected from diethylene glycol monoethyl ether, diisopropyl adipate, isopropyl myristate, isopropyl palmitate, lauryl lactate, dimethylpropylene urea and a mixture of propylene glycol monoesters and diesters of fatty acids.

It has been found that the TTS provides sufficient permeability of the nicotine even if no permeation enhancer is present. Therefore, in certain embodiments of the invention, the nicotine-containing layer does not comprise a permeation enhancer. In other embodiments, the nicotine-containing layer does not comprise any further excipient or additive, but is constituted of nicotine and a polymer matrix, preferably of nicotine and the silicone acrylic hybrid polymer, solely.

Fillers such as silica gels, titanium dioxide and zinc oxide may be used in conjunction with the polymer in order to influence certain physical parameters, such as cohesion and bond strength, in the desired way.

In general, it is preferred according to the invention that no further excipients or additives are required. Thus, the TTS has a structure of low complexity.

### Release Characteristics

The TTS in accordance with the invention are designed for transdermally administering nicotine to the systemic circulation for a predefined extended period of time.

In one aspect, the TTS according to the invention as described above provide a mean release rate of 2 to 60 mg/day, preferably of 5 to 45 mg/day, and more preferably of 5 to 20 mg/day, 10 to 35 mg/day or 15 to 45 mg/day over at least 6 hours, at least 8 hours, at least 12 hours, at least 16 hours or at least 24 hours of administration.

According to certain embodiments, the TTS according to the invention as described above provide a cumulative skin permeation rate of nicotine at hour 24 as measured in a Franz diffusion cell with dermatomed human skin of 5 μg/cm²-hr to 80 μg/cm²-hr, preferably of 10 μg/cm²-hr to 60 μg/cm²-hr and more preferably of 15 μg/cm²-hr to 50 μg/cm²-hr.

In certain embodiments, the transdermal therapeutic system according to the invention as described above provides a cumulative permeated amount of nicotine as measured in a Franz diffusion cell with dermatomed human skin of 0.2 mg/cm² to 2.0 mg/cm², preferably of 0.3 mg/cm² to 1.5 mg/cm², and more preferably of 0.4 mg/cm² to 1.2 mg/cm² over a time period of 24 hours.

### Method of Treatment/Medical Use

In accordance with a specific aspect of the present invention, the TTS according to the invention is for use in a method of treatment. In particular, the TTS according to the invention is for use in a method of treating a human patient.

In certain embodiments, the TTS according to the invention is for use in a method of treating nicotine addiction, in a method of smoking cessation treatment, in a method of treating Parkinson's disease or in a method of treating Alzheimer's disease.

The TTS may be further for use in a method of treatment including applying the TTS for at least 6 hours, at least 8 hours, at least 12 hours, at least 16 hours or at least 24 hours, and in particular for 6 hours, 8 hours, 12 hours, 16 hours or 24 hours.

Accordingly, the invention is also related to TTS for use in a method of treatment, and in particular for use in a method of treating nicotine addiction, in a method of smoking cessation treatment, in a method of treating Parkinson's disease or in a method of treating Alzheimer's disease, in an around-the-clock treatment with e.g. a three-times-a-day TTS exchange mode, a twice-a-day TTS exchange mode or a once-a-day TTS exchange mode (dosing intervals of 8 hours, 12 hours or of 24 hours), or in periodic treatments, e.g. in the daytime (e. g. wherein the TTS is applied for 6 hours, 8 hours, 12 hours or 16 hours).

In accordance with another specific aspect, the present invention is also related to a method of treatment, and in particular a method of treating a human patient.

The invention is in particular related to a method of treatment, including applying a transdermal therapeutic system according to the invention to the skin of a patient.

The invention is in particular related to a method of treating nicotine addiction, a method of smoking cessation treatment, a method of treating Parkinson's disease or a method of treating Alzheimer's disease, including applying a transdermal therapeutic system according to the invention to the skin of a patient.

The method of treatment as outlined above may in particular include applying a transdermal therapeutic system according to the invention for at least 6 hours, at least 8 hours, at least 12 hours, at least 16 hours or at least 24 hours to the skin of a patient.

The method of treatment as outlined above may also include applying a transdermal therapeutic system according to the invention for 6 hours, 8 hours, 12 hours, 16 hours or 24 hours to the skin of a patient.

Accordingly, the invention is also related to a method of treatment in an around-the-clock treatment with a three-times-a-day TTS exchange mode, a twice-a-day TTS exchange mode or a once-a-day TTS exchange mode (dosing interval of 8 hours, 12 hours or 24 hours), or in periodic treatments, e.g. in the daytime (e.g. wherein the TTS is applied for 6 hours, 8 hours, 12 hours or 16 hours).

The inventors have surprisingly shown that a relatively constant nicotine blood plasma concentration can be maintained for an extended period of time by transdermal delivery of nicotine.

### Process of Manufacture

The invention further relates to a process of manufacture of a nicotine-containing layer for use in a transdermal therapeutic system and a corresponding nicotine-containing layer structure and a corresponding TTS.

The inventive process for manufacturing the nicotine-containing pressure-sensitive adhesive layer comprises the steps of:
1) combining at least the components nicotine and a silicone acrylic hybrid polymer in a solvent to obtain a coating composition;
2) coating the coating composition onto the backing layer or release liner; and
3) drying the coated coating composition to form the nicotine-containing pressure-sensitive adhesive layer.

In this process of manufacture, preferably in step 1) the nicotine is dissolved to obtain a coating composition.

In the above described process, preferably the solvent is selected from alcoholic solvents, in particular methanol, ethanol, isopropanol and mixtures thereof, and from non-alcoholic solvents, in particular ethyl acetate, hexane, n-heptane, petroleum ether, toluene, and mixtures thereof, and more preferably is selected from hexane.

By using hexane or other solvents with a low boiling temperature or other volatile solvents, the drying temperature can be kept low, thus avoiding substantial loss of active during drying.

Thus, in preferred embodiments, a silicone acrylic hybrid polymer solution in ethyl acetate is dried and the obtained silicone acrylic hybrid polymer is dissolved in n-hexane prior to step 1) in the above process.

In other preferred embodiments, in step 1) of the process above, the silicone acrylic hybrid polymer is provided in dry from or as a solution in ethyl acetate, in hexane or in n-heptane, and preferably in hexane.

In certain embodiments, the silicone acrylic hybrid polymer is provided as a solution in ethyl acetate, hexane, n-heptane, methanol or ethanol with a solids content of from 30 to 70% by weight. Preferably, the silicone acrylic hybrid polymer is provided as a solution in hexane with a solids content of from 40 to 60% by weight.

In step 3), drying is performed preferably at a temperature of from 0 to 50° C. and more preferably from 20 to 40° C., in particular at 30° C. or at room temperature.

### EXAMPLES

The present invention will now be more fully described with reference to the accompanying examples. It should be understood, however, that the following description is illustrative only and should not be taken in any way as a restriction of the invention. Numerical values provided in the examples regarding the amount of ingredients in the composition or the area weight may vary slightly due to manufacturing variability.

### Example 1

### Coating Composition

The formulations of the nicotine-containing coating composition of Example 1 and of Reference Example-1c are summarized in Table 1.1 below. The formulations are based on weight percent as also indicated in Table 1.1.

TABLE 1.1

| | Ex. 1 | |
|---|---|---|
| Ingredient (Trade Name) | Amt [g] | Solids [%] |
| Nicotine base | 1.57 | 8.0 |
| Silicone acrylic hybrid pressure-sensitive | 18.10* | 92.0 |
| adhesive in ethyl acetate. (Dow-Corning ® | | |
| 7-6102) | | |
| n-Hexane | 10.00 | — |
| Total | 29.67 | 100.0 |

| | |
|---|---|
| Area Weight [g/m²] | 112.0 |
| Nicotine content [mg/cm²] | 0.896 |

*Amount given as solids amount as measured after evaporation of solvent

### Preparation of the Coating Composition

A beaker was loaded with the silicone acrylic hybrid pressure-sensitive adhesive solution and the solvent evaporated. The silicone acrylic hybrid pressure-sensitive adhesive was redissolved in n-hexane, the nicotine base was added and the mixture was then stirred until a homogeneous mixture was obtained (stirring time is 3 hours or longer for re-dissolving the silicone acrylic hybrid pressure-sensitive adhesive throughout the examples, if not indicated otherwise).

### Coating of the Coating Composition

The resulting nicotine-containing coating composition was coated on a polyethylene terephthalate film (fluorine polymer coated, 75 μm thickness, which may function as release liner) and dried for approx. 4 min at room temperature. The coating thickness gave an area weight of the nicotine-containing pressure sensitive adhesive layer of 112.0 g/m². The dried film was laminated with a polyethylene terephthalate backing layer (15 μm thickness) to provide a nicotine-containing self-adhesive layer structure.

### Preparation of the TTS

The individual systems (TTS) were then punched out from the nicotine-containing self-adhesive layer structure. In specific embodiments a TTS as described above can be provided with a further self-adhesive layer of larger surface area, preferably with rounded corners, comprising a pressure-sensitive adhesive matrix layer which is free of active agent. This is of advantage when the TTS, on the basis of its physical properties alone, does not adhere sufficiently to the skin and/or when the nicotine-containing matrix layer, for the purpose of avoiding waste, has pronounced corners (square or rectangular shapes). The TTS are then punched out and sealed into pouches of the primary packaging material.

### Measurement of Skin Permeation Rate

The permeated amount and the corresponding skin permeation rates of (i) a TTS prepared according to Example 1 and (ii) a NicoDerm® CQ® patch (21 mg) were determined by in vitro experiments in accordance with the OECD Guideline (adopted Apr. 13, 2004) carried out with a 10 ml Franz diffusion cell. NicoDerm® CQ® (21 mg/24 h) is a commercially available TTS with a nicotine content of 5.182 mg/cm² and a patch size of 22 cm².

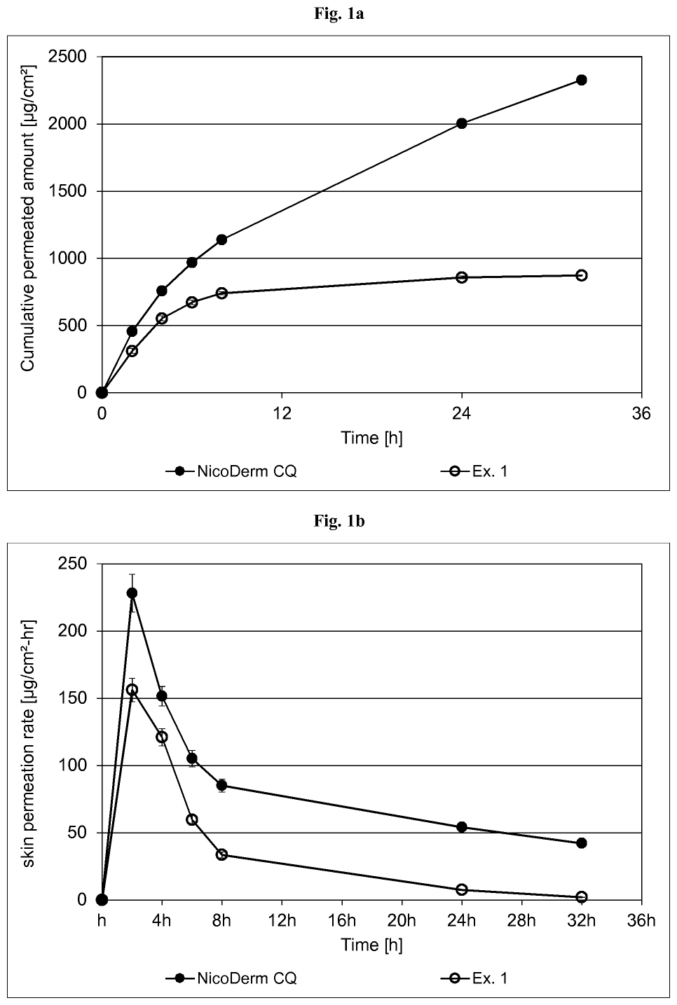

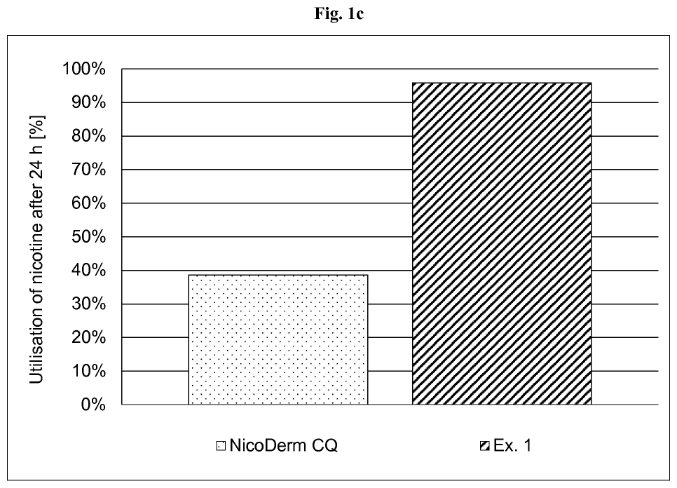

Split thickness human skin (male abdomen, year of birth 1979) was used. A dermatome was used to prepare skin to a thickness of 800 μm, with an intact epidermis for all TTS. Die cuts with an area of 0.814 cm² were punched from the TTS. The nicotine permeated amount in the receptor medium of the Franz cell (phosphate buffer solution pH 5.5 with 0.1% sodium azide as antibacteriological agent) at a temperature of 32±1° C. was measured and the corresponding skin permeation rate calculated. The results are shown in Tables 1.2 and 1.3 and FIGS. 1a and 1b.

TABLE 1.2
Cumulative permeated amount with SD [μg/cm²]

| | | |
|---|---|---|
| Elapsed | Ex. 1 (n = 3) | NicoDerm ® CQ ® (n = 3) |

| time [h] | Amount | SD | Amount | SD |
|---|---|---|---|---|
| 0 | 0 | 0 | 0 | 0 |
| 2 | 312.33 | 17.47 | 456.33 | 28.04 |
| 4 | 554.33 | 17.56 | 759.33 | 26.27 |
| 6 | 673.67 | 18.18 | 969.67 | 33.02 |
| 8 | 740.53 | 16.51 | 1139.67 | 35.16 |
| 24 | 858.20 | 10.81 | 2003.33 | 26.31 |
| 32 | 873.77 | 10.43 | 2327.50 | 31.82 |

TABLE 1.3
Skin permeation rate with SD [μg/cm²-hr]

| | | |
|---|---|---|
| Elapsed | Ex. 1 (n = 3) | NicoDerm ® CQ ® |

| time [h] | Rate | SD | Rate | SD |
|---|---|---|---|---|
| 0 | 0 | 0 | 0 | 0 |
| 2 | 156.17 | 8.74 | 228.17 | 14.02 |
| 4 | 121.00 | 6.26 | 151.50 | 7.26 |
| 6 | 59.67 | 3.18 | 105.17 | 6.11 |
| 8 | 33.43 | 2.22 | 85.00 | 4.77 |
| 24 | 7.35 | 0.37 | 53.98 | 1.23 |
| 32 | 1.95 | 0.11 | 42.00 | 1.06 |

### Utilization of Nicotine

The utilization of nicotine at 24 h was calculated based on the cumulative permeated amount at 24 h and the initial nicotine content. The results are shown in Table 1.4 and in FIG. 1c.

TABLE 1.4
Utilization of nicotine after 24 h [%]

| Example 1 | NicoDerm ® CQ ® |
|---|---|
| (n = 3) | (n = 3) |
| 96 | 39 |

### Examples 2A to 2D

### Coating Composition

The formulations of the nicotine-containing coating compositions of Examples 2a to 2d are summarized in Tables 2.1 below. The formulations are based on weight percent, as also indicated in Table 2.1.

TABLE 2.1

| | Ex. 2a | | Ex. 2b | | Ex. 2c | | Ex. 2d | |
|---|---|---|---|---|---|---|---|---|
| | Amt | Solids | Amt | Solids | Amt | Solids | Amt | Solids |
| Ingredient (Trade Name) | [g] | [%] | [g] | [%] | [g] | [%] | [g] | [%] |
| | | | | | Layer 1 | | | |
| Nicotine base | 1.63 | 8.1 | 1.18 | 5.9 | 1.65 | 8.0 | 1.65 | 8.0 |
| Silicone acrylic hybrid | 18.41* | 91.9 | 18.80* | 94.1 | 19.02* | 92.0 | 19.02* | 92.0 |
| pressure-sensitive adhesive | | | | | | | | |
| in ethyl acetate. (Dow- | | | | | | | | |
| Corning ® 7-6102) | | | | | | | | |
| n-Hexane | 10.04 | — | 10.03 | — | 10.13 | — | 10.13 | — |
| Total | 30.08 | 100.0 | 30.01 | 100.0 | 30.8 | 100.0 | 30.8 | 100.0 |

| Area Weight [g/m²] | 150 | 234 | 113 | 130 |
|---|---|---|---|---|
| Nicotine content [mg/cm²] | 1.215 | 1.381 | 0.904 | 1.040 |
| | | | Layer 2 Solids [%] | |
| Silicone acrylic hybrid | | | 100.0 | 0.0 |
| pressure-sensitive adhesive | | | | |
| in n-heptane. Solids content | | | | |
| of 50% by weight (Dow- | | | | |
| Corning ® 7-6101) | | | | |
| Silicone acrylic hybrid | | | 0.0 | 100.0 |
| pressure-sensitive adhesive | | | | |
| in ethyl acetate. Solids | | | | |
| content of 50% by weight | | | | |
| (Dow-Corning ® 7-6102) | | | | |
| Area Weight [g/m²] | | | 115.0 | 105.0 |
| Nicotine content [mg/cm²] | | | 0.000 | 0.000 |

*Amount given as solids amount as measured after evaporation of solvent

### Preparation of the Coating Composition

The coating composition of Examples 2a and 2b as well as of layer 1 of Examples 2c and 2d was prepared as outlined for Example 1.

For layer 2 of Examples 2c and 2d, the silicone acrylic hybrid pressure-sensitive adhesive solution as provided by the manufacturer (Dow-Corning® 7-6101 for Example 2c and Dow-Corning® 7-6102 for Example 2d) was used as coating composition.

### Coating of the Coating Composition, Examples 2a and 2b

For Examples 2a and 2b, the resulting nicotine-containing coating composition was coated on a polyethylene terephthalate film (fluorine polymer coated, 75 μm thickness, which may function as release liner) and dried for approx. 6 min at room temperature. The coating thickness gave an area weight of the nicotine-containing pressure sensitive adhesive layer of 150 g/m² (Example 2a) and 234 g/m² (Example 2b), respectively. The dried film was laminated with a polyethylene terephthalate backing layer (15 pin thickness) to provide a nicotine-containing self-adhesive layer structure.

### Coating of the Coating Composition, Examples 2c and 2d

For Examples 2c and 2d, the resulting nicotine-containing coating compositions (layer 1) were coated on a polyethylene terephthalate backing layer (15 μm thickness) and dried for approx. 10 min at room temperature. The coating thickness gave an area weight of the nicotine-containing pressure sensitive adhesive layer (layer 1) of 113 g/m² (Example 2c) and 130 g/m² (Example (2d). The layer 2 coating compositions, i.e. the silicone acrylic hybrid pressure-sensitive adhesive solutions, were coated on a polyethylene terephthalate film (fluorine polymer coated, 75 μm thickness, which may function as release liner, for Example 2c, or siliconised, 100 μm thickness, which may function as release liner, for Example 2d) and dried for approx. 10 min at room temperature, followed by 10 min. at 80° C. The coating thickness gave an area weight of layer 2 of 115 g/m² (Example 2c) and 105 g/m² (Example 2d). The adhesive site of the nicotine-containing layer (layer 1) was laminated on the adhesive site of the coated and dried nicotine-free layer (layer 2) resulting in a nicotine-containing self-adhesive layer structure.

### Preparation of the TTS

The individual systems (TTS) were then punched out from the nicotine-containing self-adhesive layer structure. In specific embodiments a TTS as described above can be provided with a further self-adhesive layer of larger surface area, preferably with rounded corners, comprising a pressure-sensitive adhesive matrix layer which is free of active agent. This is of advantage when the TTS, on the basis of its physical properties alone, does not adhere sufficiently to the skin and/or when the nicotine-containing matrix layer, for the purpose of avoiding waste, has pronounced corners (square or rectangular shapes). The TTS are then punched out and sealed into pouches of the primary packaging material.

### Measurement of Skin Permeation Rate

The permeated amount and the corresponding skin permeation rates of (i) TTS prepared according to Examples 2a to 2d as well as (ii) a Nicorette® patch and a Nicotinell® patch were determined by in vitro experiments in accordance with the OECD Guideline (adopted Apr. 13, 2004) carried out with a 10 ml Franz diffusion cell. Nicorette® is a commercially available TTS based on polyacrylates with a nicotine content of 1.750 mg/cm². Nicotinell® is a commercially available TTS based on polyacrylates and contains nicotine in an amount of 1.75 mg/cm². Die cuts with a nicotine content of 2.5 mg/cm² used for the measurement of the permeation rate of Nicotinell® were obtained from the commercially available patch Nicotinell® by punching die cuts with an area of 1.188 cm² from the central area of the patch.

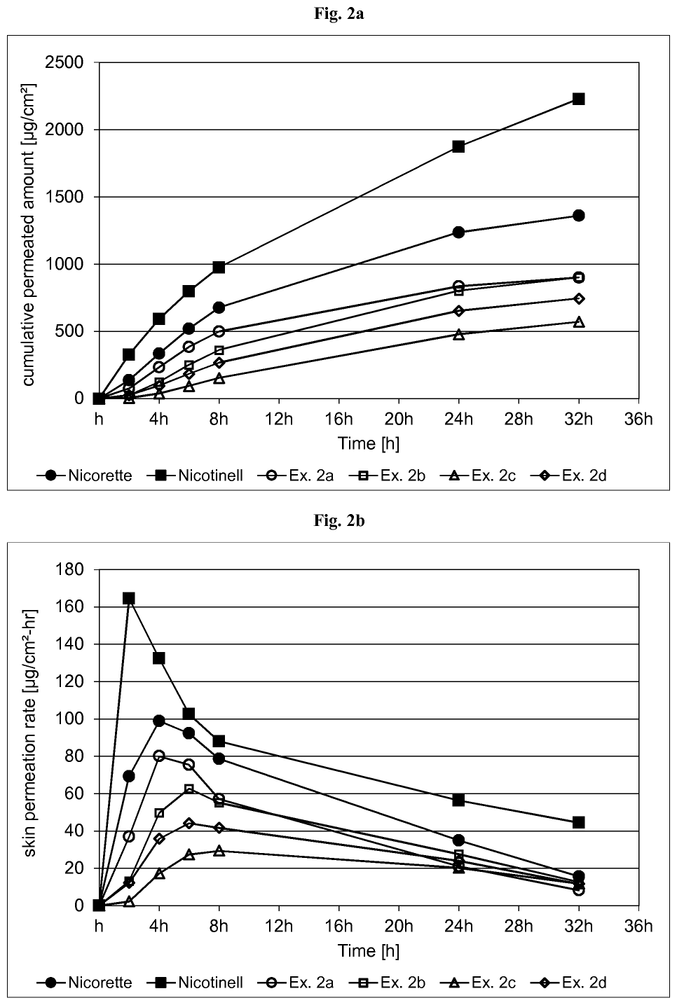

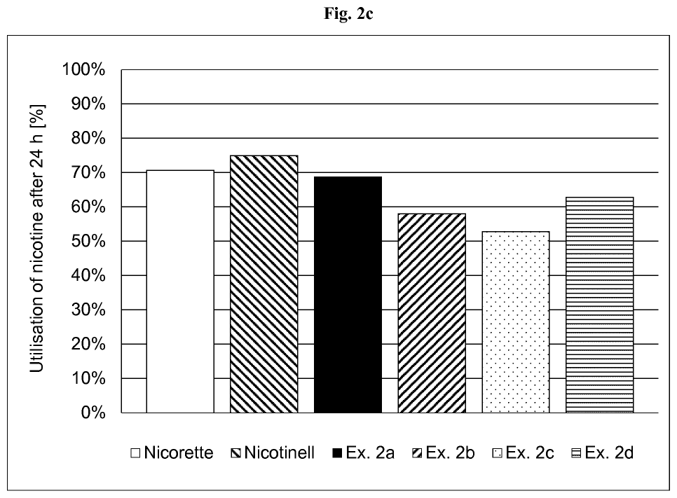

Split thickness human skin (female abdomen, date of birth 1970) was used. A dermatome was used to prepare skin to a thickness of 800 μm, with an intact epidermis for all TTS. Die cuts with an area of 1.188 cm² were punched from the TTS. The nicotine permeated amount in the receptor medium of the Franz cell (phosphate buffer solution pH 5.5 with 0.1% saline azide as antibacteriological agent) at a temperature of 32±1° C. was measured and the corresponding skin permeation rate calculated. The results are shown in Tables 2.2 to 2.5 and FIGS. 2a and 2b.

TABLE 2.2
Cumulative permeated amount with SD [μg/cm²]

| | Ex. 2a | | Ex. 2b | | Ex. 2c | | Ex. 2d | |
|---|---|---|---|---|---|---|---|---|
| Elapsed | (n = 3) | | (n = 3) | | (n = 3) | | (n = 3) | |
| time [h] | Amount | SD | Amount | SD | Amount | SD | Amount | SD |
| 0 | 0 | 0 | 0 | 0 | 0 | 0 | 0 | 0 |
| 2 | 74.07 | 33.95 | 26.37 | 4.98 | 4.46 | 0.26 | 24.33 | 5.39 |
| 4 | 234.07 | 65.05 | 125.83 | 13.56 | 38.86 | 1.59 | 96.10 | 11.91 |
| 6 | 384.73 | 70.27 | 251.17 | 12.10 | 93.50 | 2.36 | 184.33 | 16.90 |
| 8 | 498.73 | 65.50 | 361.50 | 8.60 | 152.23 | 2.94 | 267.77 | 20.15 |
| 24 | 834.40 | 26.52 | 801.50 | 22.29 | 477.56 | 4.60 | 653.10 | 16.11 |
| 32 | 901.07 | 18.24 | 902.37 | 24.01 | 571.43 | 7.62 | 745.60 | 15.07 |

TABLE 2.3
Cumulative permeated amount with SD [μg/cm²]

| | | |
|---|---|---|
| | Nicorette ® | Nicotinell ® |
| Elapsed | (n = 3) | (n = 3) |

| time [h] | Amount | SD | Amount | SD |
|---|---|---|---|---|
| 0 | 0 | 0 | 0 | 0 |
| 2 | 138.30 | 136.76 | 329.00 | 60.75 |
| 4 | 335.97 | 241.90 | 593.67 | 74.27 |
| 6 | 520.63 | 309.05 | 799.00 | 89.31 |
| 8 | 677.63 | 342.19 | 975.00 | 97.89 |
| 24 | 1236.30 | 279.90 | 1874.33 | 111.46 |
| 32 | 1360.47 | 231.02 | 2229.67 | 110.70 |

TABLE 2.4
Skin permeation rate with SD [μg/cm²-hr]

| | Ex. 2a | | Ex. 2b | | Ex. 2c | | Ex. 2d | |
|---|---|---|---|---|---|---|---|---|
| Elapsed | (n = 3) | | (n = 3) | | (n = 3) | | (n = 3) | |
| time [h] | Rate | SD | Rate | SD | Rate | SD | Rate | SD |
| 0 | 0 | 0 | 0 | 0 | 0 | 0 | 0 | 0 |
| 2 | 37.03 | 16.97 | 13.18 | 2.49 | 2.23 | 0.13 | 12.17 | 2.70 |
| 4 | 80.00 | 15.60 | 49.73 | 4.37 | 17.20 | 0.72 | 35.88 | 3.28 |
| 6 | 75.33 | 2.84 | 62.67 | 3.18 | 27.32 | 0.77 | 44.12 | 3.59 |
| 8 | 57.00 | 2.60 | 55.17 | 3.40 | 29.37 | 0.43 | 41.72 | 1.78 |
| 24 | 20.98 | 2.44 | 27.50 | 1.57 | 20.33 | 0.34 | 24.08 | 0.96 |
| 32 | 8.33 | 1.07 | 12.61 | 0.23 | 11.73 | 0.38 | 11.56 | 0.62 |

TABLE 2.5
Skin permeation rate with SD [μg/cm²-hr]

| | | |
|---|---|---|
| | Nicorette ® | Nicotinell ® |
| Elapsed | (n = 3) | (n = 3) |

| time [h] | Rate | SD | Rate | SD |
|---|---|---|---|---|
| 0 | 0 | 0 | 0 | 0 |
| 2 | 69.15 | 68.38 | 164.50 | 30.38 |
| 4 | 98.83 | 54.21 | 132.33 | 7.75 |
| 6 | 92.33 | 34.32 | 102.67 | 7.52 |
| 8 | 78.50 | 17.30 | 88.00 | 4.44 |
| 24 | 34.92 | 4.12 | 56.21 | 1.08 |
| 32 | 15.52 | 6.16 | 44.42 | 0.38 |

### Utilization of Nicotine

The utilization of nicotine at 24 h was calculated based on the cumulative permeated amount at 24 h and the initial nicotine content. The results are shown in Table 2.6 and in FIG. 2c.

TABLE 2.6
Utilization of nicotine after 24 h [%]

| Exam- | Exam- | Exam- | Exam- | | |
|---|---|---|---|---|---|
| ple 2a | ple 2b | ple 2c | ple 2d | Nicorette ® | Nicotinell ® |
| (n = 3) | (n = 3) | (n = 3) | (n = 3) | (n = 3) | (n = 3) |
| 69 | 58 | 53 | 63 | 71 | 75 |

### The Invention Relates in Particular to the Following Further Items

1. Transdermal therapeutic system for the transdermal administration of nicotine comprising a nicotine-containing layer structure, said nicotine-containing layer structure comprising:
A) a backing layer; and
B) a nicotine-containing layer;
   wherein the transdermal therapeutic system comprises a silicone acrylic hybrid polymer, and wherein the nicotine-containing layer comprises at least 0.8 mg/cm² nicotine.
   2. Transdermal therapeutic system according to item 1,
   wherein the silicone acrylic hybrid polymer is a silicone acrylic hybrid pressure-sensitive adhesive.
   3. Transdermal therapeutic system according to item 1 or 2,
   wherein the nicotine-containing layer is a matrix layer, and preferably is a nicotine-containing pressure-sensitive adhesive layer.
   4. Transdermal therapeutic system according to any one of items 1 to 3,
   wherein the nicotine-containing layer comprises:
1. nicotine; and
2. the silicone acrylic hybrid polymer.
   5. Transdermal therapeutically system according to any one of items 1 to 4,
   wherein the nicotine-containing layer structure is a nicotine-containing self-adhesive layer structure.
   6. Transdermal therapeutic system according to any one of items 1 to 5,
   wherein the nicotine-containing layer structure contains a therapeutically effective amount of nicotine.
   7. Transdermal therapeutic system according to any one of items 1 to 6,
   wherein the nicotine-containing layer comprises at least 0.90 mg/cm², preferably at least 0.95 mg/cm², more preferably at least 1.15 mg/cm² nicotine, and/or
   wherein the nicotine-containing layer comprises less than 5.0 mg/cm², less than 4.0 mg/cm², less than 3.0 mg/cm² or less than 2.0 mg/cm² nicotine.
   8. Transdermal therapeutic system according to any one of items 1 to 7,
   wherein the area weight of the nicotine-containing layer is at least 80 g/m² or is at least 90 g/m² or ranges from 80 to 300 g/m², preferably from 90 to 270 g/m², and more preferably from 100 to 230 g/m².
   9. Transdermal therapeutic system according to any one of items 1 to 8,
   wherein the amount of nicotine in the nicotine-containing layer ranges from 2 to 15%, preferably from 3 to 12% and more preferably from 4 to 10% of the nicotine-containing layer.
   10. Transdermal therapeutic system according to any one of items 1 to 9,
   wherein the amount of nicotine contained in the transdermal therapeutic system ranges from 5 to 90 mg, preferably from 8 to 75 mg, and most preferably from 10 to 60 mg.
   11. Transdermal therapeutic system according to any one of items 1 to 10,
   wherein the nicotine-containing layer structure does not comprise an additional skin contact layer.
   12. Transdermal therapeutic system according to any one of items 1 to 10,
   wherein the nicotine-containing layer structure comprises an additional skin contact layer,
   wherein the additional skin contact layer comprises or does not comprise nicotine, and preferably does not comprise nicotine.
   13. Transdermal therapeutic system according to item 12,
   wherein the additional skin contact layer is a pressure-sensitive adhesive layer comprising a silicone acrylic hybrid polymer, a non-hybrid polymer and preferably a non-hybrid pressure-sensitive adhesive, or any mixture thereof.
   14. Transdermal therapeutic system according to any one of items 1 to 13,
   wherein the nicotine in the nicotine-containing layer is included in the form of the free base.
   15. Transdermal therapeutic system according to any one of items 1 to 14,
   wherein the nicotine-containing layer is obtainable by incorporating the nicotine in the form of the free base.
   16. Transdermal therapeutic system according to any one of items 1 to 15,
   wherein at least 90 mol %, preferably at least 95 mol %, more preferably at least 98 mol % and most preferably at least 99 mol % of the nicotine in the nicotine-containing layer is present in the form of the free base.
   17. Transdermal therapeutic system according to any one of items 1 to 16,
   wherein the nicotine in the nicotine-containing layer is completely dissolved, or
   wherein the nicotine-containing layer contains nicotine droplets, preferably constituted of nicotine free base.
   18. Transdermal therapeutic system according to any one of items 1 to 17,
   wherein the nicotine-containing layer is a nicotine-containing pressure-sensitive adhesive layer comprising the silicone acrylic hybrid polymer,
   wherein the amount of the silicone acrylic hybrid polymer ranges from 55 to 98%, preferably from 70 to 97% or from 80 to 96% by weight based on the total weight of the nicotine-containing layer.
   19. Transdermal therapeutic system according to any one of items 1 to 18,
   wherein the silicone acrylic hybrid polymer is a silicone acrylic hybrid pressure-sensitive adhesive obtainable from
(a) a silicon-containing pressure-sensitive adhesive composition comprising acrylate or methacrylate functionality.
   20. Transdermal therapeutic system according to any one of items 1 to 19,
   wherein the silicone acrylic hybrid polymer is a silicone acrylic hybrid pressure-sensitive adhesive comprising the reaction product of
(a) a silicon-containing pressure-sensitive adhesive composition comprising acrylate or methacrylate functionality;
(b) an ethylenically unsaturated monomer; and
(c) an initiator.
   21. Transdermal therapeutic system according to item 19 or 20,
   wherein the silicon-containing pressure-sensitive adhesive composition comprising acrylate or methacrylate functionality comprises the condensation reaction product of
(a1) a silicon resin,
(a2) a silicone polymer, and
(a3) a silicon-containing capping agent comprising acrylate or methacrylate functionality.
   22. Transdermal therapeutic system according to any one of items 19 to 21,
   wherein the silicon-containing pressure-sensitive adhesive composition comprising acrylate or methacrylate functionality comprises the condensation reaction product of
(a1) a silicon resin,
(a2) a silicone polymer, and
(a3) a silicon-containing capping agent comprising acrylate or methacrylate functionality, wherein said silicone-containing capping agent is of the general formula XYR′_{b}SiZ_{3-b}, wherein X is a monovalent radical of the general formula AE, where E is —O— or —NH— and A is an acryl group or methacryl group, Y is a divalent alkylene radical having from 1 to 6 carbon atoms, R′ is a methyl or a phenyl radical, Z is a monovalent hydrolysable organic radical or halogen, and b is 0 or 1;
   wherein the silicone resin and silicone polymer are reacted to form a pressure-sensitive adhesive, wherein the silicone-containing capping agent is introduced prior to, during, or after the silicone resin and silicone polymer are reacted,
   and wherein the silicone-containing capping agent reacts with the pressure-sensitive adhesive after the silicone resin and silicone polymer have been condensation reacted to form the pressure-sensitive adhesive, or the silicone-containing capping agent reacts in situ with the silicone resin and silicone polymer.
   23. Transdermal therapeutic system according to any one of items 20 to 22,
   wherein the ethylenically unsaturated monomer is selected from the group consisting of aliphatic acrylates, aliphatic methacrylates, cycloaliphatic acrylates, cycloaliphatic methacrylates, and combinations thereof, each of said compounds having up to 20 carbon atoms in the alkyl radical.
   24. Transdermal therapeutic system according to any one of items 20 to 23,
   wherein the reaction product of
(a) the silicon-containing pressure-sensitive adhesive composition comprising acrylate or methacrylate functionality;
(b) the ethylenically unsaturated monomer; and
(c) the initiator
   contains a continuous, silicone external phase and a discontinuous, acrylic internal phase.
   25. Transdermal therapeutic system according to any one of items 20 to 23,
   wherein the reaction product of
(a) the silicon-containing pressure-sensitive adhesive composition comprising acrylate or methacrylate functionality;
(b) the ethylenically unsaturated monomer; and
(c) the initiator
   contains a continuous, acrylic external phase and a discontinuous, silicone internal phase.
   26. Transdermal therapeutic system according to any one of items 1 to 18,
   wherein the silicone acrylic hybrid polymer comprises a reaction product of a silicone polymer, a silicone resin and an acrylic polymer, wherein the acrylic polymer is covalently self-crosslinked and covalently bound to the silicone polymer and/or the silicone resin.
   27. Transdermal therapeutic system according to any one of items 1 to 26,
   wherein the silicone acrylic hybrid polymer contains a continuous, silicone external phase and a discontinuous, acrylic internal phase.
   28. Transdermal therapeutic system according to any one of items 1 to 27,
   wherein the silicone acrylic hybrid polymer contains a continuous, acrylic external phase and a discontinuous, silicone internal phase.
   29. Transdermal therapeutic system according to any one of items 1 to 28,
   wherein the nicotine-containing layer comprises or does not comprise a non-hybrid polymer,
   wherein the non-hybrid polymer preferably is a non-hybrid pressure-sensitive adhesive,
   wherein the non-hybrid polymer is preferably selected from polysiloxanes, polyisobutylenes, styrene-isoprene-styrene block copolymers and acrylic polymers.
   30. Transdermal therapeutic system according to item 29,
   wherein the non-hybrid polymer is selected from acrylic polymers.
   31. Transdermal therapeutic system according to item 30,
   wherein the non-hybrid polymer is selected from acrylic polymers comprising functional groups or comprising no functional groups.
   32. Transdermal therapeutic system according to any one of items 29 to 31,
   wherein the nicotine-containing layer is a nicotine-containing pressure-sensitive adhesive layer comprising the silicone acrylic hybrid polymer,
   wherein the total polymer content in the nicotine-containing layer ranges from 75 to 98%, preferably from 80 to 98% and more preferably from 85 to 98% of the nicotine-containing layer.
   33. Transdermal therapeutic system according to any one of items 1 to 32,
   wherein the area of release ranges from 2 to 100 cm², preferably from 5 to 50 cm², and more preferably from 7 to 30 cm².
   34. Transdermal therapeutic system according to any one of items 1 to 33,
   wherein the nicotine-containing layer does not comprise a permeation enhancer.
   35. Transdermal therapeutic system according to any one of items 1 to 34,
   wherein the nicotine-containing layer does not comprise any further excipient or additive.
   36. Transdermal therapeutic system according to any one of items 1 to 35,
   wherein the nicotine-containing layer comprises further excipients or additives selected from the group consisting of cross-linking agents, solubilizers, fillers, tackifiers, film forming agents, plasticizers, stabilizers, softeners, substances for skincare, permeation enhancers, pH regulators, and preservatives.
   37. Transdermal therapeutic system according to item 36,
   wherein the tackifier is selected from triglycerides, dipropylene glycol, resins, resin esters, terpenes and derivatives thereof, ethylene vinyl acetate adhesives, dimethylpolysiloxanes and polybutenes.
   38. Transdermal therapeutic system according to item 36,
   wherein the stabilizer is selected from tocopherol and ester derivatives thereof, ascorbic acid and ester derivatives thereof, butyl hydroxyl anisol and butyl hydroxy toluene.
   39. Transdermal therapeutic system according to item 36,
   wherein the permeation enhancer is selected from diethylene glycol monoethyl ether, diisopropyl adipate, isopropyl myristate, isopropyl palmitate, lauryl lactate, dimethylpropylene urea and a mixture of propylene glycol monoesters and diesters of fatty acids.
   40. Transdermal therapeutic system according to any one of items 1 to 39,
   wherein the transdermal therapeutic system provides a mean release rate of 2 to 60 mg/day, preferably of 5 to 45 mg/day, and more preferably of 5 to 20 mg/day, 10 to 35 mg/day or 15 to 45 mg/day over at least 6 hours, at least 8 hours, at least 12 hours, at least 16 hours or at least 24 hours of administration.
   41. Transdermal therapeutic system according to any one of items 1 to 40,
   providing a cumulative skin permeation rate of nicotine at hour 24 as measured in a Franz diffusion cell with dermatomed human skin of 5 μg/cm²-hr to 80 μg/cm²-hr, preferably of 10 μg/cm²-hr to 60 μg/cm²-hr and more preferably of 15 μg/cm²-hr to 50 μg/cm²-hr.
   42. Transdermal therapeutic system according to any one of items 1 to 41,
   providing a cumulative permeated amount of nicotine as measured in a Franz diffusion cell with dermatomed human skin of 0.2 mg/cm² to 2.0 mg/cm², preferably of 0.3 mg/cm² to 1.5 mg/cm², and more preferably of 0.4 mg/cm² to 1.2 mg/cm² over a time period of 24 hours.
   43. Transdermal therapeutic system according to any one of items 1 to 42,
   further comprising a release liner and/or an adhesive overlay.
   44. Transdermal therapeutic system according to any one of items 1 to 43,
   wherein the backing layer is substantially nicotine-impermeable.
   45. Transdermal therapeutic system according to any one of items 1 to 44 for use in a method of treatment.
   46. Transdermal therapeutic system according to item 45
   for use in a method of treating nicotine addiction, in a method of smoking cessation treatment, in a method of treating Parkinson's disease or in a method of treating Alzheimer's disease.
   47. Transdermal therapeutic system according to item 45 or 46
   for use in a method of treatment including applying the transdermal therapeutic system for at least 6 hours, at least 8 hours, at least 12 hours, at least 16 hours or at least 24 hours.
   48. Transdermal therapeutic system according to item 47
   for use in a method of treatment including applying the transdermal therapeutic system for 6 hours, 8 hours, 12 hours, 16 hours or 24 hours.
   49. A method of treatment
   including applying a transdermal therapeutic system according to any one of items 1 to 44 to the skin of a patient.
   50. A method of treating nicotine addiction and/or a method of smoking cessation treatment, a method of treating Parkinson's disease or a method of treating Alzheimer's disease,
   including applying a transdermal therapeutic system according to any one of items 1 to 44 to the skin of a patient.
   51. A method of treatment according to item 49 or 50
   including applying a transdermal therapeutic system according to any one of items 1 to 44 for at least 6 hours, at least 8 hours, at least 12 hours, at least 16 hours or at least 24 hours to the skin of a patient.
   52. A method of treatment according to any one of items 49 to 51
   including applying a transdermal therapeutic system according to any one of items 1 to 44 for 6 hours, 8 hours, 12 hours, 16 hours or 24 hours to the skin of a patient.
   53. A process for manufacturing a nicotine-containing pressure-sensitive adhesive layer comprising the steps of:
1) combining at least the components nicotine and a silicone acrylic hybrid polymer in a solvent to obtain a coating composition;
2) coating the coating composition onto a backing layer or a release liner; and
3) drying the coated coating composition to form the nicotine-containing pressure-sensitive adhesive layer.
   54. The process according to item 53,
   further comprising the step of drying a silicone acrylic hybrid polymer solution in ethyl acetate and dissolving the obtained silicone acrylic hybrid polymer in hexane prior to step 1).
   55. The process according to item 53, wherein the silicone acrylic hybrid polymer is provided in dry from or as a solution in ethyl acetate, in hexane or in n-heptane, preferably in hexane.
   56. The process according to any one of items 53 to 55,
   wherein the solvent is selected from alcoholic solvents, in particular methanol, ethanol, isopropanol and mixtures thereof, and from non-alcoholic solvents, in particular ethyl acetate, hexane, n-heptane, petroleum ether, toluene, and mixtures thereof, and more preferably is selected from hexane.
   57. Transdermal therapeutic system for the transdermal administration of nicotine comprising a nicotine-containing self-adhesive layer structure comprising:
A) a backing layer;
B) a nicotine-containing pressure-sensitive adhesive layer comprising:
   1. nicotine included in the form of the free base in an amount of 4% to 10% of the nicotine-containing pressure-sensitive adhesive layer; and
   2. a silicone acrylic hybrid pressure-sensitive adhesive in an amount of from 90 to 96% of the nicotine-containing pressure-sensitive adhesive layer;
      wherein the area weight of the nicotine-containing pressure-sensitive adhesive layer ranges from 100 to 250 g/m²,
      wherein the silicone acrylic hybrid pressure-sensitive adhesive in the nicotine-containing pressure-sensitive adhesive layer contains a continuous, silicone external phase and a discontinuous, acrylic internal phase,
      wherein the self-adhesive layer structure does not comprise an additional skin contact layer.
      58. Transdermal therapeutic system for the transdermal administration of nicotine comprising a nicotine-containing self-adhesive layer structure comprising:
A) a backing layer;
B) a nicotine-containing pressure-sensitive adhesive layer comprising:
   1. nicotine included in the form of the free base in an amount of 6% to 10% of the nicotine-containing pressure-sensitive adhesive layer; and
   2. a silicone acrylic hybrid pressure-sensitive adhesive in an amount of from 90 to 94% of the nicotine-containing pressure-sensitive adhesive layer;
      wherein the area weight of the nicotine-containing pressure-sensitive adhesive layer ranges from 90 to 150 g/m², and
      wherein the silicone acrylic hybrid pressure-sensitive adhesive in the nicotine-containing pressure-sensitive adhesive layer contains a continuous, silicone external phase and a discontinuous, acrylic internal phase,
      wherein the self-adhesive layer structure comprises an additional skin contact layer with an area weight of from 80 to 140 g/m².

1. Transdermal therapeutic system for the transdermal administration of nicotine comprising a nicotine-containing layer structure, said nicotine-containing layer structure comprising:
A) a backing layer; and
B) a nicotine-containing layer;
wherein the transdermal therapeutic system comprises a silicone acrylic hybrid polymer, and
wherein the nicotine-containing layer comprises at least 0.8 mg/cm² nicotine.

2. Transdermal therapeutic system according to claim 1,
wherein the silicone acrylic hybrid polymer is a silicone acrylic hybrid pressure-sensitive adhesive.

3. Transdermal therapeutic system according to claim 1 or 2,
wherein the nicotine-containing layer is a matrix layer, and preferably is a nicotine-containing pressure-sensitive adhesive layer.

4. Transdermal therapeutic system according to any one of claims 1 to 3,
wherein the nicotine-containing layer comprises:
1. nicotine; and
2. the silicone acrylic hybrid polymer.

5. Transdermal therapeutic system according to any one of claims 1 to 4,
wherein the nicotine-containing layer comprises at least 0.90 mg/cm², preferably at least 0.95 mg/cm², more preferably at least 1.15 mg/cm² nicotine, and/or
wherein the nicotine-containing layer comprises less than 5.0 mg/cm², less than 4.0 mg/cm², less than 3.0 mg/cm², less than 2.0 mg/cm² or less than 1.7 mg/cm² nicotine.

6. Transdermal therapeutic system according to any one of claims 1 to 5,
wherein the area weight of the nicotine-containing layer is at least 80 g/m² or is at least 90 g/m² or ranges from 80 to 300 g/m², preferably from 90 to 270 g/m², and more preferably from 100 to 230 g/m².

7. Transdermal therapeutic system according to any one of claims 1 to 6,
wherein the amount of nicotine in the nicotine-containing layer ranges from 2 to 15%, preferably from 3 to 12% and more preferably from 4 to 10% of the nicotine-containing layer, and/or
wherein the amount of nicotine contained in the transdermal therapeutic system ranges from 5 to 90 mg, preferably from 8 to 75 mg, and most preferably from 10 to 60 mg.

8. Transdermal therapeutic system according to any one of claims 1 to 7,
wherein the nicotine-containing layer structure does not comprise an additional skin contact layer, or
wherein the nicotine-containing layer structure comprises an additional skin contact layer, and
wherein the additional skin contact layer comprises or does not comprise nicotine, and preferably does not comprise nicotine, and
wherein the additional skin contact layer is a pressure-sensitive adhesive layer comprising a silicone acrylic hybrid polymer, a non-hybrid polymer and preferably a non-hybrid pressure-sensitive adhesive, or any mixture thereof.

9. Transdermal therapeutic system according to any one of claims 1 to 8,
wherein the silicone acrylic hybrid polymer is a silicone acrylic hybrid pressure-sensitive adhesive obtainable from
(a) a silicon-containing pressure-sensitive adhesive composition comprising acrylate or methacrylate functionality.

10. Transdermal therapeutic system according to any one of claims 1 to 9,
wherein the silicone acrylic hybrid polymer is a silicone acrylic hybrid pressure-sensitive adhesive comprising the reaction product of
(a) a silicon-containing pressure-sensitive adhesive composition comprising acrylate or methacrylate functionality;
(b) an ethylenically unsaturated monomer; and
(c) an initiator,
wherein preferably the silicon-containing pressure-sensitive adhesive composition comprising acrylate or methacrylate functionality comprises the condensation reaction product of
(a1) a silicon resin,
(a2) a silicone polymer, and
(a3) a silicon-containing capping agent comprising acrylate or methacrylate functionality.

11. Transdermal therapeutic system according to any one of claims 1 to 8,
wherein the silicone acrylic hybrid polymer comprises a reaction product of a silicone polymer, a silicone resin and an acrylic polymer, wherein the acrylic polymer is covalently self-crosslinked and covalently bound to the silicone polymer and/or the silicone resin.

12. Transdermal therapeutic system according to any one of claims 1 to 11,
wherein the silicone acrylic hybrid polymer contains a continuous, silicone external phase and a discontinuous, acrylic internal phase, or
wherein the silicone acrylic hybrid polymer contains a continuous, acrylic external phase and a discontinuous, silicone internal phase.

13. Transdermal therapeutic system according to any one of claims 1 to 12,
wherein the nicotine-containing layer comprises or does not comprise a non-hybrid polymer,
wherein the non-hybrid polymer preferably is a non-hybrid pressure-sensitive adhesive,
wherein the non-hybrid polymer is preferably selected from polysiloxanes, polyisobutylenes, styrene-isoprene-styrene block copolymers and acrylic polymers, and/or
wherein the nicotine-containing layer does not comprise a permeation enhancer, and/or
wherein the nicotine-containing layer does not comprise any further excipient or additive.

14. Transdermal therapeutic system according to any one of claims 1 to 13,
wherein the nicotine-containing layer comprises further excipients or additives selected from the group consisting of cross-linking agents, solubilizers, fillers, tackifiers, film forming agents, plasticizers, stabilizers, softeners, substances for skincare, permeation enhancers, pH regulators, and preservatives.

15. Transdermal therapeutic system according to any one of claims 1 to 14 for use in a method of treatment, and
preferably for use in a method of treating nicotine addiction, in a method of smoking cessation treatment, in a method of treating Parkinson's disease or in a method of treating Alzheimer's disease.

16. A method of treatment, and preferably a method of treating nicotine addiction and/or a method of smoking cessation treatment, a method of treating Parkinson's disease or a method of treating Alzheimer's disease,
including applying a transdermal therapeutic system according to any one of claims 1 to 14 to the skin of a patient.

17. A process for manufacturing a nicotine-containing pressure-sensitive adhesive layer comprising the steps of:
1) combining at least the components nicotine and a silicone acrylic hybrid polymer in a solvent to obtain a coating composition;
2) coating the coating composition onto a backing layer or a release liner; and
3) drying the coated coating composition to form the nicotine-containing pressure-sensitive adhesive layer.

18. The process according to claim 17,
further comprising the step of drying a silicone acrylic hybrid polymer solution in ethyl acetate and dissolving the obtained silicone acrylic hybrid polymer in hexane prior to step 1),
wherein the solvent preferably is selected from alcoholic solvents, in particular methanol, ethanol, isopropanol and mixtures thereof, and from non-alcoholic solvents, in particular ethyl acetate, hexane, n-heptane, petroleum ether, toluene, and mixtures thereof, and more preferably is selected from hexane.
